(19) United States
(12) Patent Application Publication
(10) Pub. No.: US 2002/0111360 A1
(43) Pub. Date: Aug. 15, 2002

(54) Quinazoline formulations and therapeutic use thereof

(75) Inventors: Seang H. Yiv, Encinitas, CA (US); Mingshu Li, St. Paul, MN (US); Fatih M. Uckun, White Bear Lake, MN (US)

Correspondence Address: MERCHANT & GOULD PC, P.O. BOX 2903, MINNEAPOLIS, MN, 55402-0903, US

(73) Assignee: PARKER HUGHES INSTITUTE

(21) Appl. No.: 09/960,464

(22) Filed: Sep. 19, 2001

Related U.S. Application Data
(63) Continuation of application No. PCT/US00/07066, filed on Mar. 17, 2000.
(60) Provisional application No. 60/125,147, filed on Mar. 19, 1999.

(51) Int. Cl. A61K 31/517

(52) U.S. Cl. 514/259

(57) ABSTRACT

Pharmaceutical compositions for parenteral administration of poorly soluble quinazoline compounds in the form of microemulsions or micellar solutions are described. The compositions are useful in treating patients suffering from cancer or having allergic reactions.

### PRIORITY OF THE INVENTION

This application is a continuation application of international application number PCT/US00/07066 filed on Mar. 17, 2000 claiming priority under 35 U.S.C. 119 (a)-(e) to U.S. Provisional Application No. 60/125,147 filed on Mar. 19, 1999; the international application was published under PCT Article 21(2) in English as WO 00/56338.

### FIELD OF THE INVENTION

This application relates to new formulations for poorly water soluble quinazoline compounds and therapeutic methods for the treatment of cancers and treatment of allergic disorders by administering quinazoline formulations.

### BACKGROUND OF THE INVENTION

Quinazoline compounds have been suggested as useful compounds in the treatment of cell growth and differentiation characterized by activity of the human epidermal growth factor receptor type2 (HER2). See, for example, Myers et.al., U.S. Pat. No. 5,721,237. Some quinazoline derivatives have been suggested as useful ,Is anti-cancer agents for the treatment of specific receptor tyrosine kinase-expressing cancers, especially those expressing epithelial growth factor (EGF) receptor tyrosine kinase. See, for example, Barker et. al., U.S. Pat. No. 5,457,105. It is generally taught that quinazolines exert their anti-tumor effects via tyrosine kinase inhibition. However, while some quinazoline compounds inhibit the growth of brain tumor cells, others with equally potent tyrosine kinase inhibitory activity fail to do so (Naria et.al., 1998, Clin. Cancer Res. 4:1405-1414; Naria et.al., 1998, Clin. Cancer Res. 4:2463-2471).

Delivery of these quinazoline compounds to the treatment site is complicated by the fact that many quinazoline compounds are poorly water soluble. This is especially troublesome for aqueous intravenous delivery vehicles. These delivery vehicles are often unable to provide an effective dose of the poorly water soluble quinazoline compound to the treatment site.

Thus, there is a need for water soluble quinazoline formulations that are capable of delivering the quinazoline compounds to the treatment site without loss of biological activity. Novel water soluble quinazoline formulations may provide potent new treatment options for disorders such as cancers.

### SUMMARY OF THE INVENTION

A series of water soluble quinazoline formulations were prepared and analyzed for therapeutic activities, including anti-cancer activities, particularly against JAK3 receptor. The invention provides novel water soluble quinazoline formulations, as disclosed below, as well as therapeutic methods utilizing these formulations.

One aspect of the invention is a pharmaceutical composition comprising a dialkoxyquinazoline compound, or a pharmaceutically acceptable salt thereof, and a pharmaceutically acceptable non-topic lipid based carrier, diluent or vehicle.

Another aspect of the invention is a method of administering a dialkoxyquinazoline compound to a mammal, the method includes combining the dialkoxyquinazoline compound with a pharmaceutically acceptable lipid-based vehicle to form a pharmaceutical composition and administering the pharmaceutical composition to the mammal.

Another aspect of the invention is a pharmaceutical composition including a dialkoxyquinazoline compound in a salt form, PEG phospholipids and a cosolvent system.

Another aspect of the invention is a method of administering a dimethoxyquinazoline compound to a mammal. The method includes providing a pharmaceutical composition including dimethoxyquinazoline compound in the salt form, PEG phospholipids, a cosolvent system, and administering the pharmaceutical composition to the mammal.

### BRIEF DESCRIPTION OF THE DRAWINGS

FIG. 1 is a graph showing the solubility of WHI-P131 chloride as a function of PEG 300 and PEG 200 concentration.

FIG. 2 is a graph showing solubility of WHI-P131 chloride as a function of PEG2000-DPPE concentration.

FIG. 3 is a ternary phase diagram showing the location of a single phase microemulsion region.

FIG. 4 is a flow diagram of the cumulative solubilization enhancement of WHI-P131 with the formulations of the invention.

FIG. 5 is a graph showing the plasma concentration-time curves following i.v. bolus injection of WHI-P131 formulations of the invention in mice.

FIG. 6 is a graph showing mast cell inhibitory “anti-allergic” activity of the formulations of the invention in vitro.

### DETAILED DESCRIPTION OF THE INVENTION

Definitions:

The terms “quinazoline”, “quinazoline compound”, and “quinazoline derivative” are used interchangeably in this application to mean compounds of formula I.

All scientific and technical terms used in this application have meanings commonly used in the art unless otherwise specified. As used in this application, the following words or phrases have the meanings specified.

Halo is fluoro, chloro, bromo, or iodo. Alkyl, alkanoyl, etc., denote both straight and branched groups; but reference to an individual radical such as “propyl” embraces only the straight chain radical, a branched chain isomer such as “isopropyl” being specifically referred to. (C₁-C₄)alkyl includes methyl, ethyl, propyl, isopropyl, butyl, iso-butyl, and sec-butyl; (C₁-C₄)alkoxy includes methoxy, ethoxy, propoxy, isopropoxy, butoxy, iso-butoxy, and sec-butoxy; and (C₁-C₄)alkanoyl includes acetyl, propanoyl and butanoyl.

As used herein, “pharmaceutically acceptable carrier” means any material which, when combined with the compound of the invention, allows the compound to retain biological activity, such as the ability to potentiate antibacterial activity of mast cells and macrophages.

The term “conjugate” means a compound formed as a composite between two or more molecules. More specifically, in the present invention, the quinazoline derivative is bonded, for example, covalently bonded, to cell-specific targeting moieties forming a conjugate compound for efficient and specific delivery of the agent to a cell of interest.

The phrase “targeting moiety” means a molecule which serves to deliver the compound of the invention to a specific site for the desired activity. Targeting moieties include, for example, molecules that specifically bind molecules on a specific cell surface. Such targeting moieties useful in the invention include anti-cell surface antigen antibodies. Cytokines, including interleukins and factors such as granulocyte/macrophage stimulating factor (GMCSF) are also specific targeting moieties, known to bind to specific cells expressing high levels of their receptors.

The term “prodrug moiety” is a substitution group which facilitates use of a compound of the invention, for example by facilitating entry of the drug into cells or administration of the compound. The prodrug moiety may be cleaved from the compound, for example by cleavage enzymes in vivo. Examples of prodrug moieties include phosphate groups, peptide linkers, and sugars, which moieties can be hydrolyzed in vivo.

The term “inhibit” means to reduce by a measurable amount, or to prevent entirely.

The term “to treat” means to inhibit or block at least one symptom that characterizes a pathologic condition, in a mammal threatened by, or afflicted with, the condition.

Quinazoline Formulations

The invention is directed towards formulations for delivery of an effective amount of quinazoline to a treatment site. The formulations relate to pharmaceutical compositions that include a quinazoline compound or pharmaceutically acceptable salt thereof, and a pharmaceutically acceptable, lipid-based vehicle or delivery system. Preferably, the vehicle or delivery system of the quinazoline composition is a nontoxic delivery system or vehicle for parenteral administration. The formulations disclosed enhance the water solubility of quinazoline compounds without loss of biologic activity of the quinazoline compound at the treatment site.

Quinazoline Compounds

Quinazoline compounds include quinazolines having the formula:

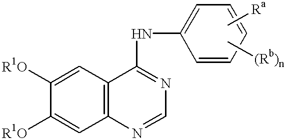

where:

R^{a} is hydrogen; halo; hydroxy; mercapto; (C₁-C₄)hydroxyalkyl, methylenedioxy, ethylenedioxy, benzyloxy, OCF₃, SCF₃, SO₃H, SO₂F, SO₂NR²R³ in which R² is hydrogen or (C₁-C₄)alkyl and R³ is hydrogen, (C₁-C₄)alkyl, or phenyl, NR²R^{a} in which R² is as defined above and R⁴ is phenyl, or R¹ a group of the formula

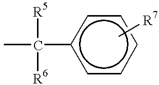

in which

R⁵ and R⁶ are each, independently, hydrogen, (C₁-C₄)alkyl, or (C₁-C₄)perfluoroalkyl, and R⁷ is hydrogen, halo, hydroxy, (C₁-C₄)alkyl, (C₁-C₄)alkoxy, (C₁-C₄)hydroxyalkyl, or N(R²)₂ in which R² is as defined above;

n is an integer of 1-4;

R^{b} is each, independently, hydrogen; halo; hydroxy; mercapto; (C₁-C₄)alkyl, (C₁-C₄)alkoxy, (C₁-C₄)thioalkyl, (C₁-C₄)hydroxyalkyl, nitro, cyano, methylenedioxy, ethylenedioxy, COCH₃, CF₃; OCF₃; SCF₃; COOH; SO₃H; SO₂F; phenyl or phenyl substituted by a group selected from halo, hydroxy, mercapto, (C₁-C₄)alkyl, (C₁-C₄)alkoxy, (C₁-C₄)thioalkyl, (C₁-C₄)hydroxyalkyl, amino, nitro, cyano, CF₃, COOH, SO₃H, SO₂NR²R³ in which R² and R³ are as defined below, and SO₂F;

R^{a} is also benzyloxy substituted on the phenyl portion by a group defined above, NR²R³ in which R² is H or (C₁-C₄)alkyl and R³ is H, (C₁-C₄)alkyl, phenyl or phenyl substituted by a group as defined above;

R¹ is (C₁-C₄)alkyl, preferably methyl, or a pharmaceutically acceptable salt thereof, such as an acid addition salt.

Preferred quinazoline compounds useful in the treatment of tumors are described more fully below and particularly in the Examples.

Delivery System

The quinazoline compounds of the invention are useful as pharmaceutical compositions prepared with a therapeutically effective amount of a quinazoline compound and a pharmaceutically acceptable carrier. The quinazoline formulations of the invention can be administered to a mammalian host, such as a human patient in a variety of forms adapted to the chosen route of administration, i.e., orally or parenterally, by intravenous, intramuscular, transdermal or subcutaneous routes. The present invention is especially suitable for parenteral administration, particularly intravenous administration. The amount of quinazoline compounds in such therapeutically useful formulations is such that an effective dosage level will be obtained.

The quinazoline formulations may be administered intravenously or intraperitoneally by infusion or injection. Solutions of the quinazoline compounds can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders including the quinazoline compounds which are adapted for extemporaneous preparation of sterile injectable or infusible solutions or dispersions, or encapsulated in liposomes. Preferably, the vehicle is a micellar solution, microemulsion or mixtures thereof. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, such as microemulsions, or by the use of surfactants, such as micellar solutions.

Micellar Systems

Micelles are composed of aggregates consisting of generally 50 or more surfactant molecules. Micelles form in aqueous solutions at surfactant concentrations above the critical micellar concentration (CMC). Micelles have the ability to solubilize lipophilic or amphiphilic compounds. Thus, micellar systems can be used to enhance the solubility of poorly water soluble substances, such as some quinazoline compounds.

As illustrated in the Examples, a number of micellar solutions are good solubilizing vehicles for poorly water soluble quinazoline compounds. Micellar system formulations include a quinazoline compound, one or more surfactants, and a carrier.

Surfactants such as PEGylated phosphatidylethanolamines (1,2-Dipalmitoyl-sn-Glycero-3-Phosphoethanolamine-N-[Poly(ethylene glycol) 5000] and 1,2-Dipalmitoyl-sn-Glycero-3-Phosphoethanolamine-N-[Poly(ethylene glycol) 2000]) are effective in enhancing the solubilization of quinazoline compounds. The solubilization enhancement, as represented by the amount of solubilized quinazoline compound (in milligram) per gram of surfactant varies with the type of surfactant used and depends on the hydrophobic chain length and polyoxyethylene number of the PEGylated phospholipid. Preferred PEGylated phospholipids include PEG2000-DPPE® and PEG5000-DPPE® and are commercially available from Avanti Polar-Lipids Inc., (Alabaster, Ala.).

The micellar solution may include a second surfactant such as, block copolymers of ethylene oxide and propylene oxide alone or in addition to the PEGylated phosphatidylethanolamine surfactant. Preferred block copolymers of ethylene oxide and propylene oxide include; Pluronic F-77, Pluronic F-87, and Pluronic F-88 and are commercially available form BASF Corp., (Mount Olive, N.J.)

The micellar solution may include a carrier. A preferred carrier is propylene glycol such as 1,2-propanediol.

Microemulsion Systems

Microemulsions are thermodynamically stable, transparent, dispersions of water and oil, stabilized by an interfacial film of surfactant molecules. Microemulsions are characterized by their submicron particle size of 0.1 μm or below. Microemulsions and self-emulsifying drug delivery systems (SEDDS) can be used to enhance the solubility of poorly water soluble substances, such as some quinazoline compounds.

As illustrated in the Examples, a number of microemulsion solutions are good solubilizing vehicles for poorly water soluble quinazoline compounds. Microemulsion system formulations include a quinazoline compound, one or more surfactants, and a carrier.

The microemulsion solution may include one or more surfactants. These include block copolymers of ethylene oxide and propylene oxide. Preferred block copolymers of ethylene oxide and propylene oxide include; Pluronic F-77, Pluronic F-87, and Pluronic F-88 and are commercially available from BASF Corp., (Mount Olive, N.J.)

Other surfactants useful in microemulsion solutions include, ethoxylated castor oil such as Cremophor® EL castor oil commercially available from BASF Corp., (Mount Olive, N.J.,) and purified soy bean phospholipid or lecithins such as phosphatidylcholine or Phospholipon® 90G commercially available from American Lecithin (Oxford, Conn.)

The microemulsion solution may include one or more a carriers. Preferred carriers include, propylene glycol such as 1,2-propanediol, and medium chain triglycerides and monoglycerides such as, triglycerides of caprylic/capric acid such as, Captex® 355, Captex® 350 and Captex® 200 commercially available from Abitec Corp., (Columbus, Ohio)

The prevention of the action of microorganisms in the formulation can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the quinazoline compounds in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Methods of Treatment

The quinazoline formulations of the invention are useful for the treatment of animals, including humans. In particular, these quinazoline formulations have been found to be potent inhibitors of tumor cell proliferation and survival, and effective to induce apoptosis of malignant cells.

Compounds of the invention have surprisingly been found to be effective for inducing apoptosis and/or cytotoxicity of leukemia cells. In particular, 4-(4′-hydroxyphenyl)amino-6,7-dimethoxyquinazoline compounds (WHI-P131) of the invention have been found to effectively induce apoptosis in multi-drug resistant leukemia. WHI-P131 is also a potent inhibitor of Janus kinase 3 (JAK 3) and shows considerable clinical potential for treatment of hematologic malignancies as well as allergic disorders. A preferred compound for the treatment of multi-drug resistant leukemia is 4-(3′-bromo-4′-hydroxyphenyl)amino-6,7-dimethoxyquinazoline.

Compounds of the invention that are particularly useful for treating leukemia include:

4-(3′,5′-dibromo-4′-methylphenyl)amino-6,7-dimethoxyquinazoline,

4-(2′,4′,6′-tribromophenyl)amino-6,7-dimethoxyquinazoline,

4-(2′,3′,5′,6′-tetrafluoro-4′-bromophenyl)amino-6,7-dimethoxyquinazoline,

4-(4′-fluorophenyl)amino-6,7-dimethoxyquinazoline,

4-(3′-fluorophenyl)amino-6,7-dimethoxyquinazoline,

4-(2′-fluorophenyl)amino-6,7-dimethoxyquinazoline,

4-(4′-trifluoromethylphenyl)amino-6,7-dimethoxyquinazoline,

4-(2′-trifluoromethylphenyl)amino-6,7-dimethoxyquinazoline, and

4-(3′,5′-bis-trifluoromethylphenyl)amino-6,7-dimethoxyquinazoline .

Compounds of the invention that are particularly useful for treating breast tumors include:

4-(3′-bromophenyl)amino-6,7-dimethoxyquinazoline,

4-(3′,5′-dibromo-4′-hydroxyphenyl)amino-6,7-dimethoxyquinazoline,

4-(3′-chloro-4′-hydroxyphenyl)amino-6,7-dimethoxyquinazoline,

4-(3′,5′-bis-trifluoromethylphenyl)amino-6,7-dimethoxyquinazoline,

4-(2′,3′,5′,6′-tetrafluoro-4′-bromophenyl)amino-6,7-dimethoxyquinazoline,

4-(4′-fluorophenyl)amino-6,7-dimethoxyquinazoline,

4-(3′-fluorophenyl)amino-6,7-dimethoxyquinazoline, and

4-(2′-fluorophenyl)amino-6,7-dimethoxyquinazoline.

Useful dosages of the quinazoline compounds can be determined by comparing their in vitro activity, and in vivo activity in animal models. Methods for the extrapolation of effective dosages in mice, and other animals, to humans are known to the art; for example, see U.S. Pat. No. 4,938,949.

The amount of the quinazoline compounds required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The quinazoline compounds are conveniently administered in unit dosage form; for example, containing 5 to 1000 mg, conveniently 10 to 750 mg, most conveniently, 50 to 500 mg of active ingredient per unit dosage form.

Ideally, the quinazoline compounds should be administered to achieve peak plasma concentrations of from about 0.5 to about 75 μM, preferably, about 1 to 50 μM, most preferably, about 2 to about 30 μM. This may be achieved, for example, by the intravenous injection of a 0.05 to 5% solution of the quinazoline compounds. Desirable blood levels may be maintained by continuous infusion to provide about 0.01-5.0 mg/kg/hr or by intermittent infusions containing about 0.4-15 mg/kg of the quinazoline compounds.

The quinazoline compounds may conveniently be presented in a single dose or as divided doses administered at appropriate intervals, for example, as two, three, four or more sub-doses per day. The sub-dose itself may be further divided, e.g., into a number of discrete loosely spaced administrations.

Targeting quinazolines to cells

In a preferred embodiment, the quinazoline compound is targeted to cells where treatment is desired, for example, to leukemia cells, to breast cells, or to other tumor cells. The compound is targeted to the desired cell by conjugation to a targeting moiety that specifically binds the desired cell, thereby directing administration of a conjugated molecule. Useful targeting moieties are ligands which specifically bind cell antigens or cell surface ligands, for example, antibodies against the B cell antigen, CD19 (such as B43) and the like.

To form the conjugates of the invention, targeting moieties are covalently bonded to sites on the quinazoline compound. The targeting moiety, which is often a polypeptide molecule, is bound to compounds of the invention at reactive sites, including NH₂, SH, CHO, COOH, and the like. Specific linking agents are used to join the compounds. Preferred linking agents are chosen according to the reactive site to which the targeting moiety is to be attached.

Methods for selecting an appropriate linking agent and reactive site for attachment of the targeting moiety to the compound of the invention are known, and are described, for example, in Hermanson, et al., Bioconjugate Techniques, Academic Press, 1996; Hermanson, et al., Immobilized Affinity Ligand Techniques, Academic Press, 1992; and Pierce Catalog and Handbook, 1996, pp. T155-T201.

Administration of Quinazoline Formulations

According to the invention, quinazoline compounds may be administered prophylactically, i.e., prior to onset of the pathological condition, or the quinazoline compounds may be administered after onset of the reaction, or at both times.

### EXAMPLES

The invention may be further clarified by reference to the following Examples, which serve to exemplify some of the preferred embodiments, and not to limit the invention in any way.

### Example 1

### Synthesis of Quinazoline Derivatives

All chemicals were purchased from the Aldrich Chemical Company, Milwaukee, Wis., and were used directly for synthesis. Anhydrous solvents such as acetonitrile, methanol, ethanol, ethyl acetate, tetrahydrofuran, chloroform, and methylene chloride were obtained from Aldrich as sure seal bottles under nitrogen and were transferred to reaction vessels by cannulation. All reactions were carried out under a nitrogen atmosphere.

The key starting material, 4-chloro-6,7-dimethoxyquinazoline, was prepared according to published procedures (Nomoto, et al., 1990, Chem. Pharm. Bull., 38:1591-1595; Thomas, C. L., 1970, IN: Catalytic Processes and Proven Catalysts, Academic Press, New York, N.Y.) as outlined below in Scheme 1. Specifically, 4,5-dimethoxy-2-nitrobenzoic acid (compound 1) was treated with thionyl chloride to form acid chloride, followed by reacting with ammonia to yield 4,5-dimethoxy-2-nitrobenzamide (compound 2). Compound 2 was reduced with sodium borohydride in the presence of catalytic amounts of copper sulphate to give 4,5-dimethoxy-2-aminobenzamide (compound 3), which was directly refluxed with formic acid to yield 6,7-dimethoxyquinazoline-4(3H)-one (compound 4). Compound 4 was refluxed with phosphorus oxytrichloride to give 4-chloro-6,7-dimethoxyquinazoline (compound 5) in good yield.

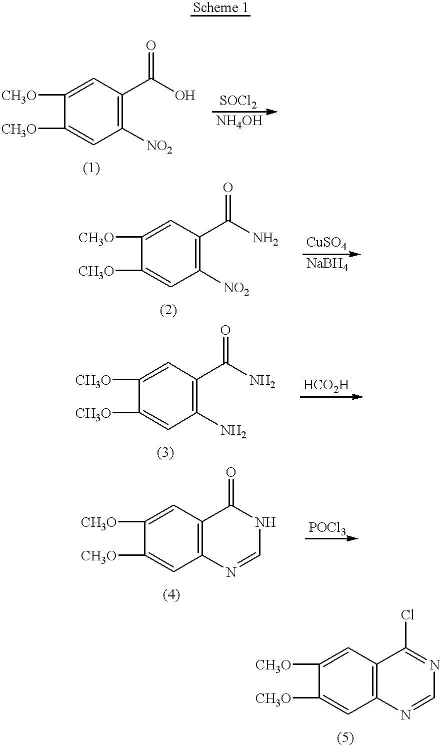

Substituted quinazoline derivatives were prepared by the condensation of 4-chloro-6,7-dimethoxyquinazoline with substituted anilines as outlined below in Scheme 2:

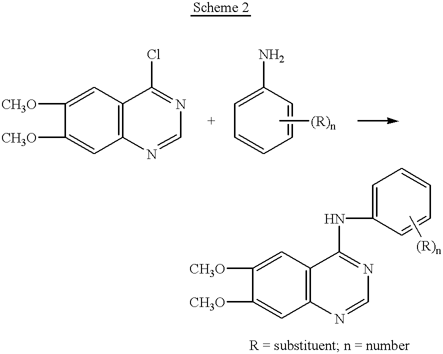

Specifically, a mixture of 4-chloro-6,7-dimethoxyquinazoline (448 mg, 2 mmols) and the substituted aniline (2.5 mmols) in EtOH (20 ml) was heated to reflux. After refluxing for 4-24 hours, an excess amount of Et₃N was added, and the solvent was concentrated to give the crude product which was recrystalized from DMF.

As discussed above, the novel hydroxy-substituted quinazoline derivatives of the invention were created by reacting the appropriate substituted anilines with the key starting material, 4-chloro-6,7-dimethoxyquinazoline.

Physical Characteristics:

Melting points are uncorrected. ¹H NMR spectra were recorded using a Varian Mercury 300 spectrometer in DMSO-d₆ or CDCl₃. Chemical shifts are reported in parts per million (ppm) with tetramethylsilane (TMS) as an internal standard at zero ppm. Coupling constants (J) are given in hertz and the abbreviations s, d, t, q, and m refer to singlet, doublet, triplet, quartet and multiplet, respectively. Infrared spectra were recorded on a Nicolet PROTEGE 460-IR spectrometer. Mass spectroscopy data were recorded on a FINNIGAN MAT 95, VG 7070E-HF G.C. system with an HP5973 Mass Selection Detector. UV spectra were recorded on BECKMAN DU 7400 and using MeOH as the solvent. TLC was performed on a precoated silica gel plate (Silica Gel KGF; Whitman Inc). Silica gel (200-400 mesh, Whitman Inc.) was used for all column chromatography separations. All chemicals were reagent grade and were purchased from Aldrich Chemical Company (Milwaukee, Wis.) or Sigma Chemical Company (St. Louis, Mo.).

### Example 2

### Bromine Substituted Quinazoline Compounds

Bromine substituted quinazoline derivatives were synthesized and characterized as discussed above in Example 1. The structures and physical data are shown below:

Bromine Substituted Quinazoline Compounds

| No | Name | Structure | Formula | MW |
|---|---|---|---|---|
| 1 | P-79 | 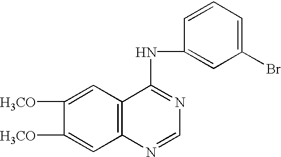 | C₁₆H₁₄BrN₃O₂ | 360 |
| 2 | P-88 | 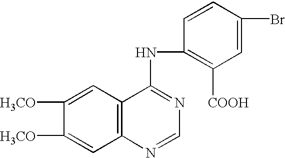 | C₁₇H₁₄BrN₃O₄ | 404 |
| 3 | P-97 | 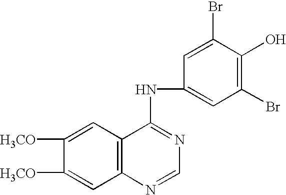 | C₁₆H₁₃Br₂N₃O₃ | 455 |
| 4 | P-111 | 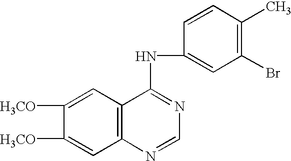 | C₁₇H₁₆BrN₃O₂ | 374 |
| 5 | P-112 | 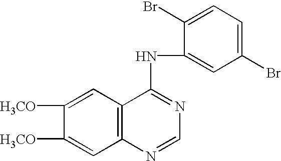 | C₁₆H₁₃Br₂N₃O₂ | 439 |
| 6 | P-154 | 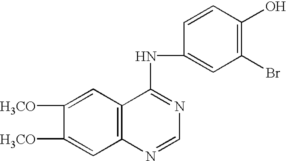 | C₁₆H₁₄BrN₃O₃ | 376 |
| 7 | P-160 | 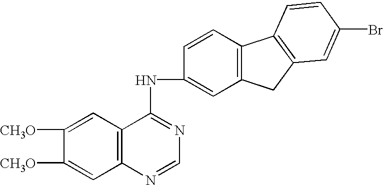 | C₂₃H₁₈BrN₃O₂ | 448 |
| 8 | P-164 | 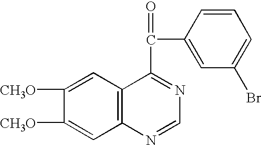 | C₁₇H₁₃BrN₂O₃ | 373 |
| 9 | P-190 | 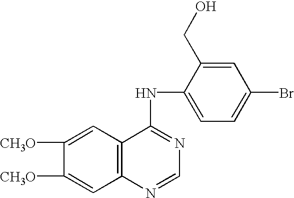 | C₁₇H₁₆BrN₃O₃ | 389 |
| 10 | P-210 | 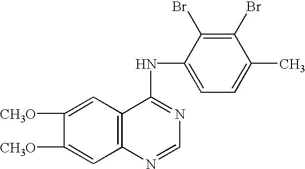 | C₁₇H₁₅Br₂N₃O₂ | 453 |
| 11 | P-211 | 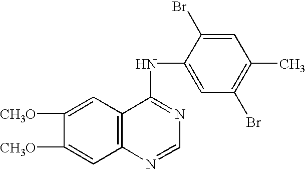 | C₁₇H₁₅Br₂N₃O₂ | 453 |
| 12 | P-212 | 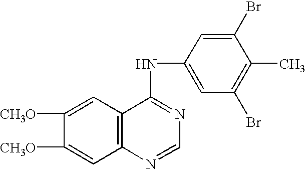 | C₁₇H₁₅Br₂N₃O₂ | 453 |
| 13 | P-214 | 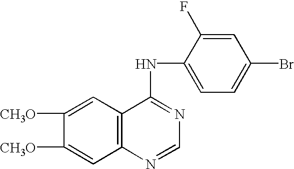 | C₁₆H₁₃BrFN₃O₂ | 378 |
| 14 | P-222 | 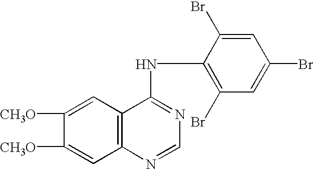 | C₁₆H₁₂Br₃N₃O₂ | 518 |
| 15 | P-234 | 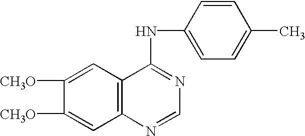 | C₁₇H₁₇N₃O₂ | 295 |
| 16 | P-241 | 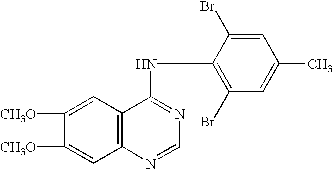 | C₁₇H₁₅Br₂N₃O₂ | 452 |
| 17 | P-258 | 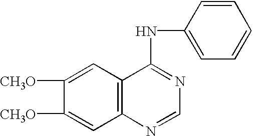 | C₁₆H₁₅N₃O₂ | 281 |
| 18 | P-260 | 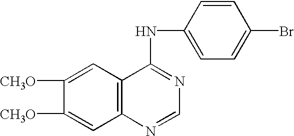 | C₁₆H₁₄BrN₃O₂ | 360 |
| 19 | P-261 | 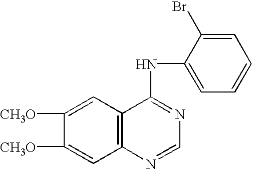 | C₁₆H₁₄BrN₃O₂ | 360 |
| 20 | P-262 | 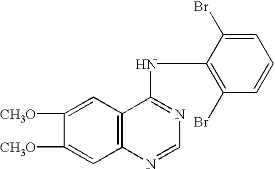 | C₁₆H₁₃Br₂N₃O₂ | 439 |
| 21 | P-263 | 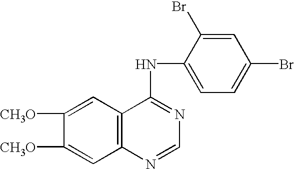 | C₁₆H₁₃Br₂N₃O₂ | 439 |

### 4-(3′-Bromophenyl)-amino-6,7-dimethoxyquinazoline (HI-P79)

Yield 84.17%; m.p.246.0-249.0° C. ¹H NMR(DMSO-d₆): δ 10.42(br, s, 1H, NH), 8.68(s, 1H , 2-H), 8.07-7.36(m, 5H, 5, 2′,4′,5′,6′-H), 7.24(s, 1H, 8H), 3.98(s, 3H, —OCH₃), 3.73(s, 3H, —OCH₃); IR(KBr)νₘₐₓ: 3409, 2836, 1632, 1512, 1443, 1243, 1068 cm⁻¹; GC/MS m/z 361(M⁺+1, 61.8), 360(M⁺, 100.0), 359(M⁺−1, 63.5), 344(11.3), 222(10.9), 140(,13.7). Anal. (C₁₆H₁₄BrN₃O₂ HCl) C, H, N.

### 4-(4′-Bromo-2′-caboxylphenyl)-amino-6,7-dimethoxyquinazoline(HI-P88)

Yield 92.82%; m.p.>300.0° C. ¹H NMR(DMSO-d₆+CF₃CO₂H): δ 9.95(d, 1H), 8.74(d, 1H, Ar—H), 8.30, 8.28(2d, 2H), 7.95(d, 1H), 7.83(s, 1H), 4.21(s,3H, —OCH₃), 4.15(s,3H, —OCH₃). UV(MeOH): 205, 229.0 nm. IR(KBr)νₘₐₓ: 3444(br), 2737, 1592, 1504, 1443, 1273, 1070 cm⁻¹. GC/MS m/z 388(M⁺+1 —OH, 18.08), 387(M⁺—OH,100.00), 386(M⁺−1 —OH, 30.84), 385(97.52), 299(4.78). Anal. (C₁₆H₁₄BrN₃O₂ HCl) C, H, N.

### 4-(3′,5′-Dibromo-4′-hydroxylphenyl)-amino-6,7-dimethoxyquinazoline (HI-P97)

Yield 72.80%; m.p.>300.0° C. ¹H NMR(DMSO-d₆): δ 9.71(s, 1H, —NH), 9.39(s, 1H, —OH), 8.48(s, 1H, 2-H), 8.07(s, 2H, 2′,6′-H), 7.76(s, 1H, 5-H), 7.17(s, 1H, 8-H), 3.94(s, 3H, —OCH₃), 3.91(s, 3H, —OCH₃). UV(MeOH): 208.0, 210.0, 245.0 , 320.0 nm; IR(KBr)νₘₐₓ: 3504(br), 3419, 2868, 1627, 1512, 1425, 1250, 1155 cm⁻¹; GC/MS m/z 456(M⁺+1, 54.40), 455(M⁺, 100.00), 454(M⁺−1, 78.01), 439(M⁺—OH, 7.96), 376(M⁺+1-Br, 9.76), 375(M⁺-Br, 10.91), 360(5.23). Anal. (C₁₆H₁₃Br₂N₃O₃) C, H, N.

### 4-(3′-Bromo-4′-methylphenyl)-amino-6,7-dimethoxyquinazoline (HI-P111)

Yield 82.22%; m.p.225.0-228° C. ¹H NMR(DMSO-d₆): δ 10.23(s, 1H, —NH), 8.62(s, 1H, 2-H), 8.06(d, 1H, J_{2′,6′}=2.1 Hz, 2′-H), 7.89(s, 1H, 5-H), 7.71(dd, 1H, J_{5′,6′}=8.7 Hz, J_{2′,6′}=2.1 Hz, 6′-H), 7.37(d, 1H, J_{5′,6′}=8.7 Hz, 5′-H), 7.21(s, 1H, 8-H), 3.96(s, 3H, —OCH₃), 3.93(s, 3H, —OCH₃). UV(MeOH): 204.0, 228.0, 255.0, 320.0 nm. IR(KBr)νₘₐₓ: 3431, 3248, 2835, 1633, 1517, 1441, 1281, 1155 cm⁻¹. GC/MS m/z 375(M⁺+1, 76.76), 374(M⁺, 100.00), 373(M⁺−1, 76.91), 358(M⁺+1—OH, 11.15), 357(1.42), 356(6.31). Anal. (C₁₇H₁₆BrN₃O₂.HCl) C, H, N.

### 4-(2′,5′-Dibromophenyl)-amino-6,7-dimethoxyquinazoline (HI-P112)

Yield 70.05%; m.p.>300.0° C. ¹H NMR(DMSO-d₆): δ 11.51(s, 1H, —NH), 8.76(s, 1H, 2-H), 8.21 (s, 1H, 5-H), 7.81 (d, 1 H, J_{4′,6′}=2.4 Hz, 6′-H), 7.75(d, 1H, J_{3′,4′}=8.7 Hz, 3′-H), 7.55(dd, 1H, J_{4′, 6′}=2.4 Hz, J_{3′,4′}=8.7 Hz, 4′-H), 7.33(s, 1H, 8-H), 3.98(s, 3H, —OCH₃), 3.97(s, 3H, —OCH₃). UV(MeOH): 208.0, 238.0, 330.0 rum. IR(KBr)νₘₐₓ: 3444, 2836, 1628, 1510, 1431, 1277, 1070 cm⁻¹. GC/MS m/z 440(M⁺+1, 10.12), 439(M⁺, 7.0), 438(M⁺−1, 3.63), 360(M⁺+1-Br, 99.42), 359(M⁺—Br, 20.45), 358(M⁺−1-Br, 100.00), 343(20.80), 299(8.62). Anal. (C₁₆H₁₃Br₂N₃O₂.HCl) C, H, N.

### 4-[(3′-Bromo-9′-fluorenone)-2′-]amino-6,7-dimethoxyquinazoline (HI-P119)

Yield 75.23%; m.p.255.0-257.0° C. ¹H NMR(DMSO-d₆): δ 8.77(s, 1H, —NH), 8.33(s, 1H, 2-H), 7.89(s, 1H, 5-H), 7.40(s, 1H, 8-H), 7.74-7.26(m, 6H, Ar—H), 4.12(s,3H, —OCH₃), 4.11(s,3H, —OCH₃). UV(MeOH): 205, 229.0, 251.0, 320.0 nm. IR(KBr)νₘₐₓ: 3444, 2836, 1628, 1510, 1431, 1277, 1070 cm⁻¹. GC/MS m/z 464(M⁺+2 ,40.81), 463(M⁺+1, 7.56), 462(M³⁰ , 27.26), 384(M⁺+2-Br, 69.56), 383(M⁺+1-Br, 35.50), 382(M⁺—Br, 100.00), 352(10.85), 206(26.73), 191(11.31). Anal. (C₂₃H₁₆BrN₃O₃ HCl) C, H, N.

### 4-(2′,3′,5′,6′-Tetrafluoro-4′-bromolphenyl)-amino-6,7-dime-thoxyquinazoline (HI-P144

Yield 78.24%; m.p. 180.0-182.0° C. ¹H NMR (DMS O-d₆): δ 7.78(s, 1H, 2-H), 7.53(s, 1H, 5-H), 6.79(s, 1H, 8-H), 3.81(s,3H, —OCH₃), 3.3.79(s,3 H, —OCH₃). Anal (C₁₆H₁₀BrF₄N₃O₂.HCl) C, H, N.

### 4-(3′-Bromo-4′-hydroxylphenyl)-amino-6,7-dimethoxyquinazoline (HI-P154)

Yield 89.90%; m.p.233.0-233.5° C. ¹H NMR(DMSO-d₆): δ 10.08(s, 1H, —NH), 9.38(s, 1H, —OH), 8.40(s, 1H, 2-H), 7.89(d, 1H, J_{2′,6′}=2.7 Hz, 2′-H), 7.75(s, 1H, 5-H), 7.55(dd, 1H, J_{5′,6′}=9.0Hz, J_{2′,6′}=2.7Hz, 6′-H), 7.14(s, 1H, 8-H), 6.97(d, 1H, J_{5′,6′}=9.0Hz, 5′-H), 3.92(s, 3H, —OCH₃), 3.90(s, 3H, —OCH₃). UV(MeOH): 203.0, 222.0, 250.0, 335.0 nm. IR(KBr)νₘₐₓ: 3431(br), 2841, 1624, 1498, 1423, 1244 cm⁻¹. GC/MS m/z 378(M⁺+2, 90.68), 377(M⁺+1, 37.49), 376(M³⁰ , 100.00), 360(M³⁰ , 3.63), 298(18.86), 282 (6.65). Anal. (C₁₆H₁₄BrN₃O₃.HCl) C, H, N.

### 4-[(7′-Bromofluorene)-2]-amino-6,7-dimethoxyquinazoline (HI-P160)

Yield 73.21%; m.p. 254.0-256.0° C. ¹H NMR(DMSO-d₆): δ 9.69(br, s, 1H, —NH), 8.52(s, 1H, 2-H), 8.12-7.20(m, 9H, 5, 8,1′,3′,4′,5′,6′,8′,9′-H), 3.99(s,3H, —OCH₃), 3.94(s, 3H, —OCH₃). UV(MeOH): 208.0, 223.0, 348.0 mn. IR(KBr)νₘₐₓ: 3421, 2820, 1624, 1516, 1431, 1294, 1223 cm⁻¹. GC/MS m/z 450(M⁺+2, 100), 449(M⁺+1, 35), 448(M⁺, 95), 311(25). Anal. (C₂₃H₁₈BrN₃O₂.HCl) C, H, N.

### 4-(3′-Bromobenzoyl)-6,7-dimethoxyquinazoline (HI-P164)

Yield 81.20%, m.p.258.0-263.0° C. ¹H NMR(DMSO-d₆): δ 9.25(s, 1H, 2-H), 8.14(s, 1H, 5-H), 7.92-7.43(m, 4H, 2′,4′,5′,6′-H), 7.40(s, 1H, 8-H), 4.11(s, 3H, —OCH₃), 4.00(s, 3H, —OCH₃). UV(MeOH): 203.0, 220.0 ,238.0 nm. IR(KBr)νₘₐₓ: 3432, 1664, 1504, 1431, 1230 cm⁻¹. GC/MS m/z 374(M⁺+1, 48.96), 373(M³⁰ , 34.93), 372(M⁺−1, 47.67), 357(58.74), 343(100.00 ), 293(M⁺—Br, 31.48), 189(26.27). Anal. (C₁₇H₁₃BrN₂O₃) C, H, Br, N.

### 4-(4′-Bromo-6′-hydroxymethylphenyl)-amino-6,7-dimethoxyquinazoline (HI-P190)

Yield 73.08%; m.p. 222.0-223.0° C. ¹H NMR(DMSO-d₆): δ 11.30(s, 1H, —OH), 8.22(s, 1H, —NH), 7.77-7.23(m, 5H, 5, 8, 2′, 3′, 5′-H), 4.49(s, 2H, PhCH₂—H), 4.01(s, 3H, —OCH₃), 3.90(s, 3H, —OCH₃). UV(MeOH): 207.0, 250.0, 332.0 nm. IR(KBr)νₘₐₓ: 3446, 2829, 2752, 1652, 1560, 1471, 1365, 1280 cm⁻¹. GC/MS m/z 391(M⁺+1, 29.33), 389(M³⁰ , 29.82), 360(M⁺—CH₂OH, 50.76), 358(52.39), 311(18.33), 280(43.20), 206(62.80), 191(100.00). Anal. (C₁₇H₁₆BrN₃O₃.HCl) C, H, N.

### 4-(2′,3′-Dibromo-4′-methylphenyl)-amino-6,7-dimethoxyquinazoline (HI-P210)

Yield 81.24%, mp 233.0-236.0° C., ¹H NMR(DMSO-d₆): δ 8.55(s, 1H, —NH), 8.08(s, 1H, 2-H), 7.33-7.17(m, 4H, 5,8,5′,6′-H), 3.89(s, 6H, —OCH₃), 2.35(s,3H, —CH₃). UV(MeOH): 207.0, 232.0, 247.0 , 330.0 nm. IRνₘₐₓ (KBr): 3448, 2840, 1629, 1580, 1525, 1420, 1281 cm⁻¹. GC/MS m/z 454(M⁺+1, 4.45), 453(M³⁰ , 11.31), 452(M⁺−1,4.45), 375(20.36), 374(97.59), 373( 23.55), 372(100.00), 358 (19.61), 356 (18.43). Anal. (C₁₇H₁₅ Br₂N₃O₂.HCl) C, H, N.

### 4-(2′,5′-Dibromo-4′-methylphenyl)-amino-6,7-dimethoxyquinazoline (HI-P211)

Yield 83.50%; m.p. 282.0-284.0° C. ¹H NMR(DMSO-d₆): δ 11.30(s, 1H, —NH), 8.58(s, 1H, 2-H ), 8.00(s, 1H, 5-H), 7.65(s, 1H, 6′-H), 7.60(s, 1H, 3′-H), 7.13(s, 1H, 8-H), 3.79(s, 3H, —OCH₃), 3.78(s, 3H, —OCH₃), 2.29(s, 3H, —CH₃). UV(MeOH): 207.0, 239.0, 330.0 nm. IR(KBr)νₘₐₓ: 3442, 2620, 1631, 1580, 1514, 1380, 1280 cm⁻¹. GC/MS m/z 454(M⁺+1, 5.86), 453(M³⁰ , 16.16), 452(M⁺−1, 5.35), 374(92.12), 373(23.66), 372(100.00), 358(17.68), 356(17.35). Anal. (C₁₇H₁₅Br₂N₃O₂.HCl) C, H, N.

### 4-(3′,5′-Dibromo-4′-methylphenyl)-amino-6,7-dimethoxyquinazoline (HI-P212)

Yield 83.47%; m.p. 275.0-279.0° C. ¹H NMR(DMSO-d₆): δ 11.30(s, 1H, —NH), 8.58(s, 1H, 2-H), 8.35(s, 1H, 5-H), 7.24(s, 2H, 2′,6′-H), 7.13(s, 1H, 8-H), 3.91(s, 3H, —OCH₃), 3.88(s, 3H, —OCH₃), 2.31(s, 3H, —CH₃). UV(MeOH): 237.0, 307.0, 319.0 nm. IR(KBr)νₘₐₓ: 3471, 3434, 2640, 1633, 1580, 1504, 1420, 1281 cm⁻¹. GC/MS m/z 454(M⁺+1, 5.34), 453(M³⁰ , 16.05), 452(M⁺−1, 5.87), 374(99.02), 373(26.20), 372(100.00), 358(20.39), 356(19.98), 32(8.29), 314(8.49), 206(19.02). Anal. (C₁₇H₁₅Br₂N₃O₂ HCl) C, H, N.

### 4-(2′-Fluoro-4′-bromophenyl)-amino-6,7-dimethoxyquinazoline (HI-P214)

Yield 77.21%; m.p. 243.0-245.0° C. ¹H NMR(DMSO-d₆): δ 8.57(s, 1H, 2-H), 7.91(s, 1H, 5-H), 7.57(d, 1H, 3′-H), 7.34 (m, 2H, 5′,6′-H), 7.07(s, 1H, 8-H), 3.78(s, 3H, —OCH₃), 3.77(s, 3H, —OCH₃). UV(MeOH): 204.0, 215.0, 250.0, 330.0 nm. IR(KBr)νₘₐₓ: 3431, 2629, 1633, 1580, 1511, 1420, 1278 cm⁻¹. GC/MS m/z 379(M⁺+1,34.39), 378(M⁺, 21.33), 377(M⁺−1, 39.08), 360(62.05), 359(31.58), 358(62.57), 357(19.81), 299(19.31), 298(100.00), 282(17.88), 240(28.76). Anal. (C₁₆H₁₃BrFN₃O₂ HCl) C, H, N.

### 4-(2′,4′,6′-Tribromophenyl)amino-6,7-dimethoxyquinazoline (HI-P222)

Yield 54.86%; m.p.250.0-255.0° C. ¹H NMR(DMSO-d₆): δ 8.00(s, 1H, 2-H), 7.89(s, 2H, 3′,5′-H), 7.74(s, 1H, 5- H), 7.01(s, 1H, 8-H), 3.87(s, 3H, —OCH₃), 3.86(s, 3H, —OCH₃). UV(MeOH): 209.0, 236.0, 333.0 nm. IR(KBr)νₘₐₓ: 3417, 2838, 1625, 1514, 1429, 1276, 1073 cm⁻¹. GC/MS m/z 519(M⁺+1, 18.12), 518(M³⁰ , 17.30), 517(M⁺−1, 16.63), 439(M⁺+1-Br, 99.42), 438(M⁺—Br, 95.45), 437(M⁺−1-Br, 100.00), 359(20.80), 358(18.62), 357(19.32), 281(88.98), 207(15.42). Anal. (C₁₆H₁₂Br₃N₃O₂ HCl) C, H, N.

### 4-(2′,6′-Dibromo-4′-methylphenyl)-amino-6,7-dimethoxyquinazoline (HI-P241)

Yield 79.47%, m.p. 235.0-237.0° C. ¹H NMR(DMSO-d₆): δ 9.77(s, 1H, —HN), 8.20 (s, 1H, 2-H), 7.87(s, 1H, 8-H), 7.61(s, 2H, 3′, 5′-H), 7.15(s, 1H, 5-H), 3.93(s, 6H, —OCH₃). UV( MeOH): 208.0, 245.0, 318.0, 339.0 nm. IR(KBr)νₘₐₓ: 3241, 2839, 2783, 1635, 1580, 1514, 1420, 1360, 1281 cm⁻¹. GC/MS m/z 454(M⁺+1,7.86), 453(M⁺, 56.16), 452(M⁺−1, 15.30), 374(95.12), 373(18.66), 372(100.00), 358(29.64), 356(19.36). Anal. (C₁₇H₁₅Br₂N₃O₂ HCl) C, H, N.

### 4-(4′-Bromophenyl)-amino-6,7-dimethoxyquinazoline (HI-P260)

Yield 75.28%. m.p.270.0-272.0° C. ¹H NMR(DMSO-d₆): δ 11.30(s, 1H, —NH), 8.85(s, 1H , 2-H), 8.27(s, 1H, 5-H), 7.70(s, 4H, 2′,3′,5′,6′-H), 7.32(s, 1H, 8H), 4.02(s,3H, —OCH₃). 4.00(s,3H, —OCH₃). UV(MeOH):204.0, 218.0, 252.0, 335.0 nm. IR(KBr)νₘₐₓ: 3431, 3034, 2636,1635, 1589,1514, 1435, 1284 cm⁻¹. GC/MS m/z 361 ( M⁺+1,74.00), 360(M³⁰ , 100.00), 359(M⁺−1,72.00), 358(M⁺−2, 95.00), 329 (3.20 ), 301 (13.0), 281 (21.0), 207(38.0). Anal. (C₁₆H₁₄BrN₃O₂.HCl) C, H, N.

### 4-(2′-Bromophenyl)-amino-6,7-dimethoxyquinazoline (HI-P261)

Yield 71.94%; m.p.241.0-243.0° C. ¹H NMR(DMSO-d₆): δ 11.67 (d, 1H, —NH), 8.79 (s, 1H, 2-H), 8.32 (s, 1H, 5-H), 7.86-7.38 (m, 4H, 3′,4′,5′,6′-H1), 7.40 (s, 1H, 8H), 4.01 (s,6H, —OCH₃). UV(MeOH): 204.0, 226.0, 248.0, 330.0 nm. IR(KBr)νₘₐₓ: 3454, 3032, 2638,1630, 1589,1514, 1430, 1281 cm⁻¹. GC/MS m/z 361(M⁺+1, 7.00), 360(M⁺, 5.00), 359(M⁺−1,6.00), 358(M⁺−2, 5.00), 301(13.0), 281(21.0), 280(100.00), 207(25.00). Anal (C₁₆H₁₄BrN₃O₂.HCl) C, H, N.

### 4-(2′,6′-Dibromophenyl)-amino-6,7-dimethoxyquinazoline (HI-P262)

Yield 69.45%, mp 243.0-246.0° C., ¹H NMR(DMSO-d₆): δ 11.91(d, 1H, —NH), 8.80(s, 1H, 2-H), 8.43(s, 1H, 5-H), 7.86(d, 2H, J=8.4 Hz, 3′, 5′-H), 7.49(s, 1H, 8H), 7.35(t, 1H, J=8.4 Hz, 4′-H), 4.02(s,3H, —OCH₃), 4.01(s,3H, —OCH₃ ). UV(MeOH): 208.0, 227.0, 245.0, 330.0 nm. IR(KBr)νₘₐₓ: 3454, 3032, 2638,1630, 1589,1514, 1430, 1281 cm⁻¹.

### 4-(2′,4′-Dibromophenyl)-amino-6,7-dimethoxyquinazoline (HI-P263)

Yield 70.62%; m.p.257.0-262.0° C. ¹H NMR(DMSO-d₆): δ 11.91(d, 1H, —NH), 8.79 (s, 1H, 2-H), 8.21(s, 1H, 5-H), 8.12-7.51(m, 3H, 3′,5′,6′-H), 7.35(s, 1H, 8-H), 4.01(s,3H, —OCH₃), 3.99(s, 3H, —OCH₃). UV(MeOH): 208.0, 210.0, 248.0, 330.0 nm. IR(KBr)νₘₐₓ: 3458, 3028, 2641, 1633, 1594, 1511, 1435, 1277 cm⁻¹.

### Example 3

### Chlorine Substituted Quinazoline Compounds

Chlorine substituted quinazoline derivatives were synthesized and characterized as discussed above in Example 1. The structures and physical data are shown below:

| No | Name | Structure | Formula | MW |
|---|---|---|---|---|
| 1 | P-87 | 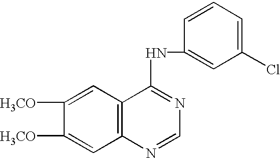 | C₁₆H₁₄ClN₃O₂ | 316 |
| 2 | P-93 | 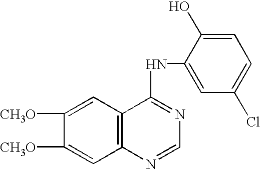 | C₁₆H₁₄ClN₃O₃ | 331 |
| 3 | P-189 | 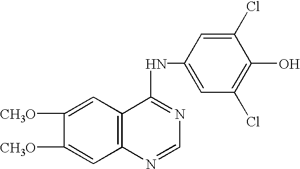 | C₁₆H₁₃Cl₂N₃O₃ | 365 |
| 4 | P-197 | 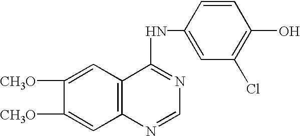 | C₁₆H₁₄ClN₃O₃ | 331 |
| 5 | P-268 | 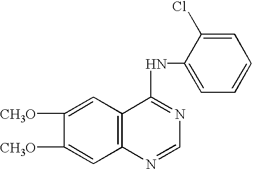 | C₁₆H₁₄ClN₃O₂ | 316 |
| 6 | P-269 | 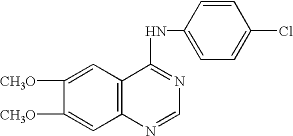 | C₁₆H₁₄ClN₃O₂ | 316 |
| 7 | P-278 | 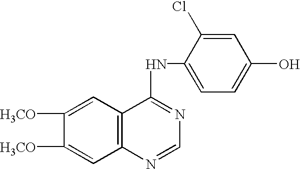 | C₁₆H₁₄ClN₃O₃ | 331 |
| 8 | P-415 | 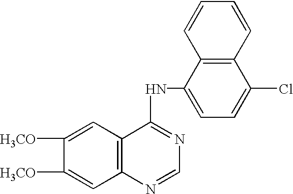 | C₂₀H₁₆ClN₃O₂ | 365 |

### 4-(3′-Chlorophenyl)-amino-6,7-dimethoxyquinazoline(HI-P87)

Yield 76.98%; m.p. 242.0-245.0° C. ¹H NMR(DMSO-d₆: δ 10.47(br, s, 1H, NH), 8.69(s, 1H, 2-H), 8.06(s, 1H, 5-H), 7.95-7.23(m, 4H,2′,4′,5′,6′-H), 7.24(s, 1H, 8-H), 3.98(s, eH, —OCH₃), 3.35(s,3H, 0 OCH₃). UV(MeOH): 228.0, 251.0, 332.0 nm. IR(KBr)νₘₐₓ: 3406, 2839, 1632, 1516, 1443, 1278, 1068 cm⁻¹. GC/MS m/z 316(M⁺−1, 68.34), 314(M⁺−2,100.00, 344(11.34), 222(4.35), 140(9.86). Found: C, 54.62; H, 4.68; N, 11.93; Cl, 19.23. C₁₆H₁₄CIN₃O₂.HCl requires: C, 54.70; H, 4.28; N, 11.96; Cl, 19.96%.

### 4-(c′-Chloroo-6′-hydroxyphenyl)amino-6,7-dimethoxyquinazoline(HI-P93)

Yield 83.08%; m.p.295.0° C.(dec). ¹H NMR9DMSO-d₆: δ 10.14(s, 1H, —OH), 8.37(s, 1H, 2-H), 7.78(s, 1H, 5H), 7.57(d, 1H, J_{2′,4′}=2.4 Hz, 2′-H),), 7.16(s, 1H, 8-H), 7.07(dd, 1H, J_{2′,4′}=2.4 Hz, J_{4′,5′}=8.7 Hz, 4′-H), 6.92(d, 1H, J_{4′,5′}=8.7 Hz, 5′-H),3.93(s,3H, —OCH₃). 3.92(s,3H, —OCH₃). UV(MeOH): 205, 229.0, 251.0, 320.0 nm. IR(KBr)νₘₐₓ: 3500(br), 3430, 2835, 1622, 1512, 1432, 1259 cm⁻¹. GC/MS m/z 333(M⁺+2, 13.41). 332(M⁺+1, 9.73, 331(M³⁰ , 39.47), 314(M⁺—OH, 100.00), 298(7.64). Found: C, 52.25; H, 4.07; N, 11.39. C₁₆H₁₄CIN₃O₃.HCl requires: C, 52.32; H, 4.09; N, 11.44%.

### 4-(4′-Hydroxyl-3′,5′-dichlorophenyl)amino-6,7-dimethoxyquinazoline(HI-P189)

Yield 79.45%; m.p. 293.0-295.0° C. ¹HNMR-DMSO-d₆): δ 11.32(s, 1H, —NH), 10.34(s, 1H, —OH), 8.87(s, 1H, 2-H), 8.29(s, 1H, 5-H), 7.90(s, 2H, 2′,6′-H), 7.32(s, 1H, 8-H), 4.01(s, 3H, —OCH₃), 3.99(s, 3H, —OCH₃). UV(MeOH): 213.0, 232.0, 250.0, 335.0 nm. IR(KBr)νₘₐₓ: 3479, 2564, 1641, 1579, 1429, 1282, 1147 cm⁻¹. GC/MS m/z 367(M⁺=2, 66.57), 366(M⁺=1, 75.91), 365(M³⁰ , 100.00), 364(M⁺−1,94.08), 349(M⁺—OH, 11.16). Anal. (C₁₆H₁₃Cl₂N₃O₃) C, H, N. Found: C,48.93; H, 4.51; N, 10.00. C₁₇ₒH₁₇Cl₂N₃O₃.Hcl requires: C, 48.80; H, 4.31; N, 10.04%.

### 4-(3′-Chloro-4′-hydroxylphenyl)-amino-6,7-dimethoxyquinazoline (HI-P197)

Yield 84.14%; m.p. 245.0° C.(dec). ¹H NMR(DMSO-d₆): Λ 10.00(s, 1H,—NH), 9.37(s, 1H, —OH), 8.41(s, 1H, 2-H), 7.78(s, 1H, 5-H), 7.49(d, 1H, J_{2′,5′}=2.7 Hz, 2′-H), 7.55(dd, 1H, J_{5′,6′}=9.0 Hz, J_{2′,6′}=2.7 Hz, 6′-H), 7.16(s, 1H, 8-H), 6.97(d, 1H, J_{5′,6′}=9.0 Hz, 5′-H), 3.93(s, 3H, —OCH₃), 3.91(s, 3H, —OCH₃). UV(MeOH): 209.0, 224.0,249.0, 330.0 nm. IR(KBr)νₘₐₓ: 3448, 2842, 1623, 1506, 1423, 1241 cm⁻¹. GC/MX m/z: 341 (M³⁰ , 100.00), 326(M⁺—CH₃, 98.50), 310(M⁺—OCH₃, 12.5), 295(9.0.), 189(13.5), 155(13.8). Found: C,521.35; H, 4.16; Cl, 19.15; N, 11.39. C₁₆H₁₄ClN₃O₃. HCl requires: C,52.32; H, 4.09; Cl, 19.07; N, 11.44%.

### 4-(2′-Chlorophenyl)-amino-6,7-dimethoxyquinazoline (HI-P268)

Yield 87.28%; m.p. 247.0-279.5° C. ¹H NMR(DMSO-d₆): Λ 11.71 (s, 1H, —NH), 8.78 (s, 1H, 2-H), 8.33 (s, 1H, 5-H), 7.67 (s, 1H, 8H), 7.68-7.42 (m, 4H, 3′,4,5,6′-H), 4.00 (s, 3H —OCH₃), 3.99(s, 3H, —OCH₃). UV(MeOH): 213.0, 234.0, 251.0, 331.0 mn. IR(KBr)νₘₐₓ: 3479, 2566, 1643, 1577, 1429, 1282, 1147cm⁻¹. GC/MX m/z 317 (M⁺+1, 6.60), 316(M³⁰ , 6.60), 315(M⁺−1, 18.52), 314(M⁺−2, 11.11), 281 (21.22), 280 (M⁺—Cl, 100.00), 264 (29.62). Found: C, 54.51; H, 4.41; N, 11.81. C₁₆H₁₄ClN₃O₂. HCl requires: C, 54.45; H, 4.26; N, 11.93%.

### 4-(4′-Chlorophenyl)-amino-6,7-dimethoxyquinazoline (HI-P269)

Yield 94.94%. m.p. 248.0-250.0° C. ¹H NMR(DMSO-d₆): Λ 11.62 (s, 1H, —NH), 8.85 (s, 1H, 2-H), 8.42 (s, 1H, 5-H), 7.88 (d, 2H, J=8.7 Hz, 3′,5′,-H), 7.54 (d, 2H, J=8.7 Hz, 2′,6′,-H), 7.38 (s, 1H, 8-H0, 4.02 (s, 3H, —OCH₃), 3.99(s, 3H, —OCH₃). UV(MeOH): 215.0, 230.0, 253.0, mn. IR(KBr)νₘₐₓ: 3477, 2563, 1640, 1578 cm⁻¹. GC/MX m/z 317 (M⁺+1,18.18), 316(M⁺,29.55), 315 (M⁺−1,48.85), 314 (M⁺−2, 61.36), 281 (32.,95), 207 (100.00). Found: C, 54.65; H, 4.38; N, 11.92. C₁₆H₁₄ClN₃O₂. HCl requires: C, 54.55; H, 4.26; N, 11.93%.

### 4-(4′-Hydroxyl-2′-chlorophenyl)-amino-6,7-dimethoxy-quinazoline (HI-P278)

Yield 81.44%; m.p. 245.0-247.0° C. ¹H NMR(DMSO-d₆): Λ 11.39(s, 1H, —NH), 10.30(s, 1H, —OH), 8.75(s, 1H, 2-H), 8.24(s, 1H, 5-H), 7.38-6.85(m, 3H, 3′,5′,6′-H), 7.37(s, 1H, 8H), 3.98(s,3H, —OCH₃), 3.96(s,3H, —OCH₃). UV(MeOH): 222.0, 234.0, 239.0, 245.0, 254.0, 348.0 nm. IR(KBr)νₘₐₓ: 3448, 3242, 3144, 3025, 2917, 2834, 1638, 1591, 1514, 1437, 1365, 1277, 1209 cm⁻¹. GC/MS m/z: 332(M⁺+1, 5.00), 331(M⁺, 17.00), 330(M⁺−1, 5.00), 297(17.00), 296(100.00), 281(18.00), 280(29.00), 253(9.00). Found: C,52.17; H,4.06;N,11.32. C₁₆H₁₄ClN₃O₃. HCl requires: C,52.32;H,4.01;N,11.44%.

### 4-(4′-Chloronaphthy-1′)-amino-6,7-dimethoxyquinazoline (HI-P415)

Yield, 85.07%

m.p. 245.0-248.0° C. ¹H NMR(DMSO-d₆): Λ 11.91(s, 1H, —NH), 8.66(s, 1H, 2-H), 8.45(s, 1H, 5-H), 8.30-7.62(m, 6H, 2′,3′,5′,6′,7′,8′-H), 7.38(s, 1H, 8-H), 4.03(s, 3H, —OCH₃), 4.01(s, 3H, —OCH₃). UV(MeOH): 211.0, 233.0, 250.0, mn. IR(KBr)νₘₐₓ: 3481, 2567, 1645, 1579cm⁻¹. Found: C, 59.32; H, 4.27; N, 10.24. C₂₀H₁₆ClN₃O₂. HCl. requires: C, 59.70; H, 4.23; N, 10.48%.

### Example 4

### Iodine Substituted Quinazoline Compounds

Iodine substituted quinazoline derivatives were synthesized as discussed above in Example 1, and analyzed. The structures and physical data are shown below:

### Iodine Substituted Quinazoline Compounds

Iodine Substituted Quinazoline Compounds

| No | Name | Structure | Formula | MW |
|---|---|---|---|---|
| 1 | P-270 | 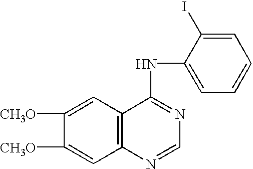 | C₁₆H₁₄IN₃O₂ | 407 |
| 2 | P-271 | 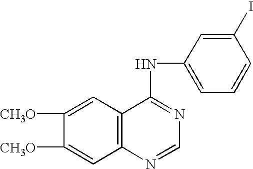 | C₁₆H₁₄IN₃O₂ | 407 |
| 3 | P-300 | 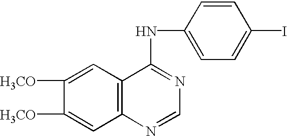 | C₁₆H₁₄IN₃O₂ | 407 |
| 4 | P-294 | 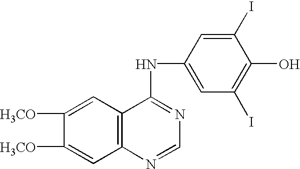 | C₁₆H₁₃I₂N₃O₃ | 549 |
| 5 | P-299 | 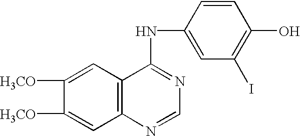 | C₁₆H₁₄IN₃O₃ | 423 |

### 4-(2′-Iodophenyl)-amino-6,7-dimethoxyquinazoline (P-270)

Yield 75.37%; m.p. 225.0-230.0° C. ¹H NMR(DMSO-d₆): δ 11.74(s, 1H, —NH), 8.79(s, 1H, 2-H), 8.33(s, 1H, 5-H), 8.05-7.13(m, 4H, 3′,4,5,6′-H), 7.44(s, 1H, 8H), 4.01(s, 6H, —OCH₃). UV(MeOH): 219.0, 222.0, 253.0, 342.0 nm. IR(KBr)νₘₐₓ: 3165, 3027, 2827, 1639, 1572, 1501, 1434, 1275, 1070 cm⁻¹. GC/MS m/z 408(M⁺+1, 3.47), 407(M⁺, 15.28), 406(M⁺−1,3.47), 281 (33.33), 280(M⁺—I, 100.00), 264(50.00), 207(34.72 ). Found: C, 43.62; H, 3.60; N, 9.42. C₁₆H₁₄IN₃O₂.HCl requires: C, 43.34; H, 3.38; N, 9.48%.

### 4-(3′-Iodophenyl)-amino-6,7-dimethoxyquinazoline (HI-P271)

Yield 79.85%; m.p. 235.0-242.0° C. ¹H NMR(DMSO-d₆): δ 11.43 (s, 1H, —NH), 8.88 (s, 1H, 2-H), 8.33 (s, 1H, 5-H), 8.13(s, 1H, 2′-H), 7.80-7.26 (m, 3H, 4′,5′,6′-H), 7.35 (s, 1H, 8H), 4.02 (s, 3H, —OCH₃), 4.00 (s, 3H, —OCH₃). UV(MeOH):.203.0, 210.0, 228.0, 251.0, 331.0 nm. (KBr)νₘₐₓ: 3191, 3022, 2940, 2836, 2576, 1629, 1516, 1444, 1276,1153, 1060 cm⁻¹. GC/MS m/z 406(M³⁰ , 1.52 ), 405(M⁺−1, 6.22), 281 (35.33), 207 (100.00). Found: C, 43.55; H, 3.43; N, 9.32. C₁₆H₁₄IN₃O₂.HCl requires: C, 43.34; H, 3.38; N, 9.48%.

### 4-(4′-Hydroxy-3,5-diiodophenyl)-amino-6,7-dimethoxy-quinazoline (HI-P294

Yield 77.47%; m.p. 259.0-260.0° C. ¹H NMR(DMSO-d₆): δ 11.13(s, 1H, NH), 9.73(s, 1H, —OH), 8.87(s, 1H, 2-H), 8.16(s, 1H, 5-H), 8.09(s, 2H, 2′,6′-H), 7.28(s, 1H, 8H), 3.98(s, 6H, —OCH₃). UV(MeOH)λₘₐₓ (ε):. 217.0 , 227.0, 252.0 nm. IR(KBr)νₘₐₓ: 3457,3201, 2934, 2832, 2566, 1629, 1562, 1521, 1439, 1275, 1075 cm⁻¹. GC/MS m/z: GC/MS m/z 422(M⁺—I, 33.53), 405(7.50), 281(86.67), 221 (51.80), 207(91.30). Found: C, 32.60; H, 2.50; N, 6.92. C₁₆H₁₃I₂N₃O₃.HCl requires: C, 32.82; H, 2.39; N, 7.18%.

### 4-(4′-Hydroxy-3′-iodophenyl)-amino-6,7-dimethoxyquinazoline(HI-P299)

Yield 71.59%; m.p. 248.0-250.0° C. ¹H NMR(DMSO-d₆): δ 11.32(d, 1H, NH), 10.62(s, 1H, —OH, 8.79(s, 1H, 2-H), 8.26(s, 1H, 5-H), 7.98-6.98(m, 3H, 2′,3′,6′-H), 7.32(s, 1H, 8H), 3.98(s, 3H, —OCH₃), 3.97(s, 3H, —OCH₃). UV(MeOH)λₘₐₓ (ε): 217.0, 227.0, 252.0 nm. IR(KBr)νₘₐₓ: 3411, 2975, 2730, 2366, 1634, 1573, 1501, 1429, 1229, 1075 cm⁻¹. GC/MS m/z: 406(M⁺−1,3.33), 405(M⁺−2, 7.50), 281 (M⁺−1-I, 26.67), 253(11.80), 207(100.00). Found: C, 41.96; H, 3.40; N, 8.98. C₁₆H₁₄IN₃O₃.HCl requires: C, 41.83; H, 3.26; N, 9.15%.

### 4-(4′-Iodophenyl)-amino-6,7-dimethoxyquinazoline (HI-P300)

Yield 85.24%; m.p. 240.0-242.0° C. ¹H NMR(DMSO-d₆): δ 11.51 (s, 1H, NH), 8.82 (s, 1H, 2-H), 8.37 (s, 1H, 5-H), 7.81 (d, 2H, J=8.4 Hz, 2′,6′- H), 7.55 (d, 2H, J=8.4 Hz, 3′,5′-H), 7.35 (s, 1H, 8H), 4.01 (s, 3H, —OCH₃), 3.98(s, 3H, —OCH₃). UV (MeOH):. 217.0, 227.0, 252.0 nm. IR (KBr)νₘₐₓ: 3211, 3032, 2832, 2720, 1629, 1573, 1501, 1434, 1235, 1153, 1070 cm⁻¹. GC/MS m/z 406(M⁺−1,3.33), 405(M⁺−2, 7.50), 281 (M⁺−1-I, 26.67), 253(11.80), 207(100.00). Found: C, 43.40; H, 3.39; N, 9.36. C₁₆H₁₄IN₃O₂.HCl. requires: C, 43.34; H, 3.38; N, 9.48%.

### Example 5

| No | Name | Structure | Formula | MW |
|---|---|---|---|---|
| 1 | P-93 | 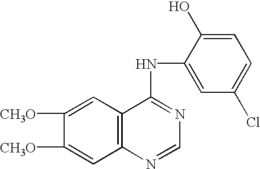 | C₁₆H₁₄ClN₃O₃ | 331 |
| 2 | P-97 | 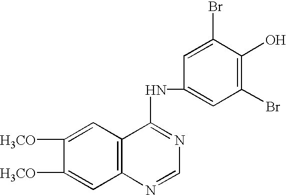 | C₁₆H₁₃Br₂N₃O₃ | 455 |
| 3 | P-131 | 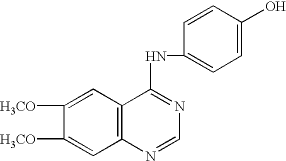 | C₁₆H₁₅N₃O₃ | 297 |
| 4 | P-132 | 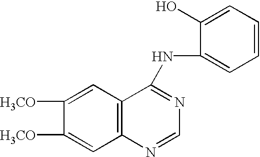 | C₁₆H₁₅N₃O₃ | 297 |
| 5 | P-133 | 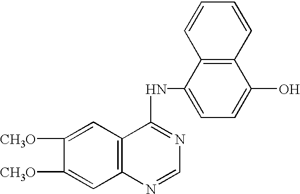 | C₁₉H₁₆N₄O₃ | 348 |
| 6 | P-150 | 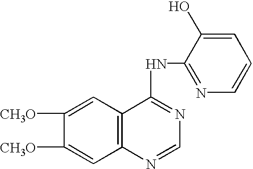 | C₁₅H₁₄N₄O₃ | 298 |
| 7 | P-154 | 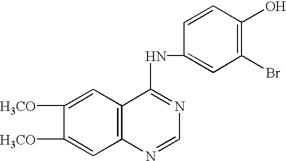 | C₁₆H₁₄BrN₃O₃ | 376 |
| 8 | P-180 | 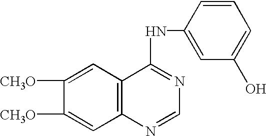 | C₁₆H₁₅N₃O₃ | 297 |
| 9 | P-182 | 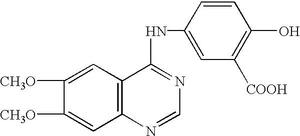 | C₁₇H₁₅N₃O₅ | 341 |
| 10 | P-189 | 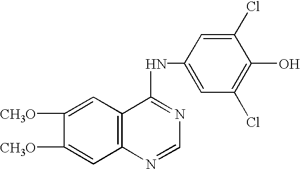 | C₁₆H₁₃Cl₂N₃O₃ | 365 |
| 11 | P-190 | 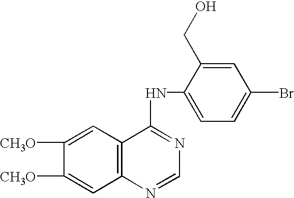 | C₁₇H₁₆BrN₃O₃ | 389 |
| 12 | P-191 | 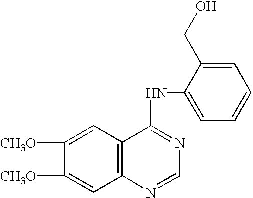 | C₁₇H₁₇N₃O₃ | 311 |
| 13 | P-192 | 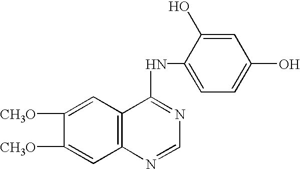 | C₁₆H₁₅N₃O₄ | 313 |
| 14 | P-197 | 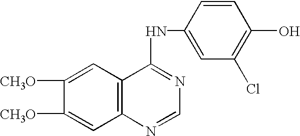 | C₁₆H₁₄ClN₃O₃ | 331 |
| 15 | P-215 | 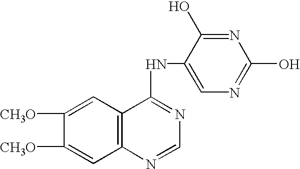 | C₁₄H₁₃N₅O₄ | 315 |
| 16 | P-259 | 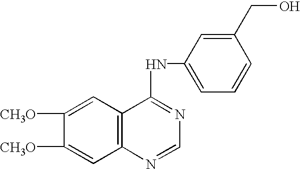 | C₁₇H₁₇N₃O₃ | 311 |
| 17 | P-265 | 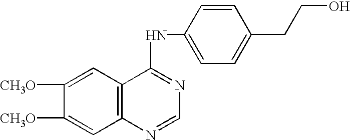 | C₁₈H₁₉N₃O₃ | 325 |
| 18 | P-266 | 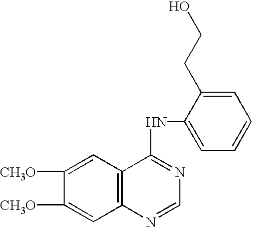 | C₁₈H₁₉N₃O₃ | 325 |
| 19 | P-274 | 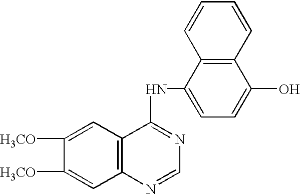 | C₂₀H₁₇N₃O₃ | 347 |
| 20 | P-275 | 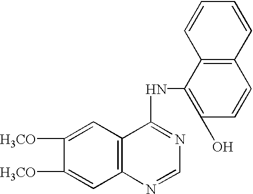 | C₂₀H₁₇N₃O₃ | 347 |
| 21 | P-276 | 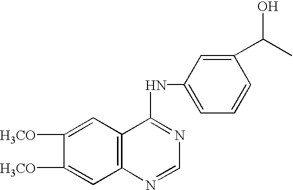 | C₁₈H₁₉N₃O₃ | 325 |
| 22 | P-277 | 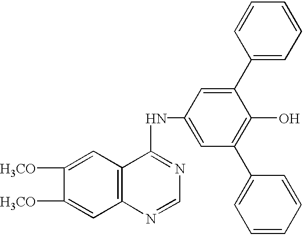 | C₂₈H₂₃N₃O₃ | 449 |
| 23 | P-278 | 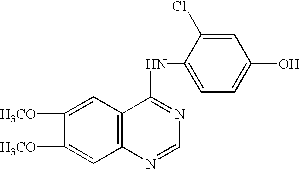 | C₁₆H₁₄ClN₃O₃ | 331 |
| 24 | P-289 | 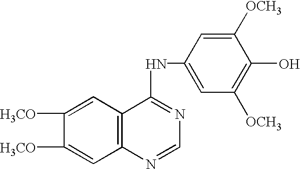 | C₁₈H₁₉N₃O₅ | 357 |
| 25 | P-292 | 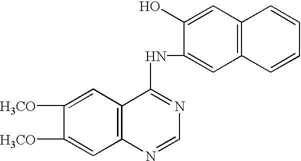 | C₂₀H₁₇N₃O₃ | 341 |
| 26 | P-293 | 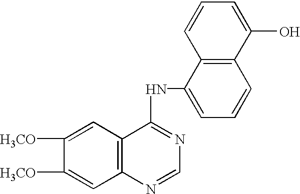 | C₂₀H₁₇N₃O₃ | 341 |
| 27 | P-294 | 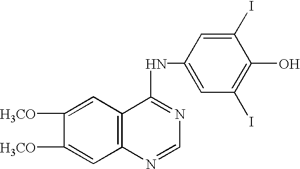 | C₁₆H₁₃I₂N₃O₃ | 549 |
| 28 | P-229 | 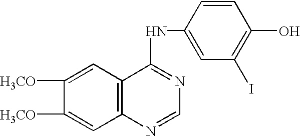 | C₁₆H₁₄IN₃O₃ | 423 |
| 29 | P-312 | 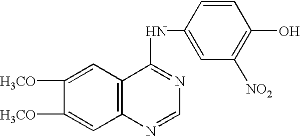 | C₁₆H₁₄N₄O₅ | 342 |
| 30 | P-313 | 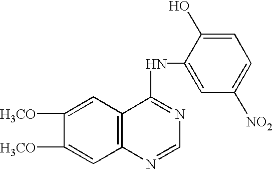 | C₁₆H₁₄N₄O₅ | 342 |
| 31 | P-315 | 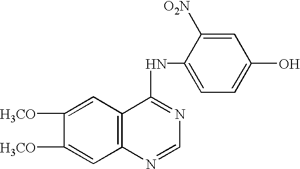 | C₁₆H₁₄N₄O₅ | 342 |
| 32 | P-323 | 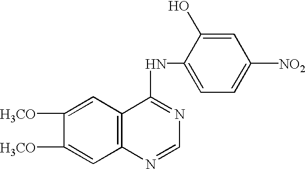 | C₁₆H₁₄N₄O₅ | 342 |

### 4-(3′-Chlooro-6′-hydroxylphenyl)amino-6,7-dimethoxyquinazoline(HI-P93)

Yield 93.08%; m.p.295.0° C.(dec). ⁻H NMR-DMSO-d₆: δ 10.14(s, 1H, —NH), 9.16(s, 1H, —OH), 8.37(s, 1H, 2-h), 7.78(s, 1H, 5H), 7.57(d. 1H, J_{2′,2′}=2.4 Hz, 2′-H),), 7.16(s, 1H, 8-H), 7.07(dd. 1H, J_{2′,4′}=2.4 Hz, J_{4′,5′}=8.7 Hz, 4′-H), 6.92(d, 1H, J_{4′,5′}−8.7 Hz, 5′-H), 3.93(s,3H, —OCH₃). 3.92(s,3H, —OCH₃. UV(MeOH): 205, 229.0, 251.0, 320.0 nm. IR(KBr)νₘₐₓ: 3500(br), 3430, 2835, 1622, 1512, 1432, 1259 cm⁻¹. GC/MS m/z 333(M⁻=2, 13.41), 332(M⁻=1, 9.73), 331(M⁺, 39.47), 314(M⁺—OH,100.00). 298(7.64). Found: C, 52.25; H, 4.07; N, 11.39, C₁₆H₁₄CIN₃O₃,HCl requires: C, 52.32; H, 4.09; N, 11.44%.

### 4-(3′,5′-Dibromo-4′-hydroxylphenyl)-amino-6,7-dimethoxyquinazoline-(HI-P97)

Yield 72.80%; m.p.>300.0° C. ¹H NMR(DMSO-d₆): δ 9.71(s, 1H, —NH), 9.39(s, 1H, —OH), 8.48(s, 1H, 2-h), 8.07(s, 2H, 2′,6′-H), 7.76(s, 1H, 5-H), 7.17(s, 1H, 8-H), 3.94(s, 3H, —OCH₃, 3.91(s, 3H, —OCH₃). UV(MeOH): 208.0, 210.0, 245.0, 320.0 nm; IR(KBr)νₘₐₓ: 3504(br), 3419, 2868, 1627, 1512, 1425, 1250, 1155 cm⁻¹; GC/MS m/z 456(M¹=1, 54.40), 455(M⁻, 100.00), 454(M⁻1, 78.01), 439(M⁻—OH, 7.96), 376(M⁻+1—Br, 9.76), 375(M⁻Br, 10.91), 360(5.23). Anal. (C₁₆H₁₃Br₂N₃O₃) C, H, N.

### 4-(4′-Hydroxylphenyl)-amino-6,7-dimethoxyquinazoline(HI-P131)

Yield 84.29%; m.p. 245.0-248.0° C. IR(KBr)νₘₐₓ: 3428, 2836, 1635, 1516, 1443, 1234 cm: ¹H NMR(DMSO-d₆: δ 11.21(s, 1H, —NH), 9.70(s, 1H, —OH), 8.74(s, 1H, 2-h), 8.22(s, 1H, 5-h), 7.40(d, 2H, J−8.9 Hz, 2′,6′-H), 7.29(s, 1H, 8-H), 6.85(d, 2H, J=8.9 Hz, 3′,5′-H), 3.98(s, 3H, —OCH₃, 3.97(s, 3H, —OCH₂). GC/MS m/z 298 (M⁻=1, 100.00), 297(M⁻, 26.6), 296(M⁺−1, 12.5). Anal. (C₁₆H₁₅N₃O₃HCl) Cl, H, N.

### 4-(2′-Hydroxylphenyl)-amino-6,7-dimethoxyquinazoline(HI-P132)

yield 82.49%; m.p. 255.0-258.0° C. IR(KBr)νₘₐₓ: 3500 (br), 3425, 2833, 1625, 1512, 1456, 1251, 1068 cm⁻¹. ¹H NMR(DMSO-d₆): δ 9.78(s, 1H, —NH), 9.29(s, 1H, —OH), 8.33(s, 1H, 2-h), 7.85(s, 1H, 5-H), 7.41-6.83(m, 4H, 3′,4′,5′,6′-H), 7.16(s, 1H, 8-H), 3.93(s, 3H, —OCH₃, 3.92(s, 3H, —OCH₃), 280(M⁺—OH, 10.0). Anal. (C₁₆H₁₅N₃O₃, HCl) C, H, N.

### 4-[(8′-Hydroxyquiline)-5′-Jamino-6,7-dimethoxyquinazoline(HI-P133)

yield 83.51%; m.p. 238.0-239.0° C. ₁H NME(DMSO-d₆: δ 10.12(br,s, 1H, —NH), 8.93-7.09 M, 8H, 2, 5, 2,2′,3′,4′,6′,7′-H), 4.04(s,3H, —OCH₃), 3.96(s,3H, —OCH₃). UV(MeOH): 204.0, 245.0, 332.0 nm. IR(KBr)νₘₐₓ: 3425(br), 2935, 1632, 1510, 1437, 1273 cm⁻¹. GC/MS m/z 349(M⁻=1,100.00), 348(m+, 26.56), 307(38.50), 289 (21.00).

### 4-[(3′-Hydroxylpyridine)-2′]-amino-6,7-dimethoxyquinazoline(HI-P150)

Yield 78.65%; m.p. 185.0-187.0° C. ¹H NMR(DMSO-d₆): δ 10.08(br,s, 1H, —NH), 8.52(s, 1H, 2-H), 7.88-7.86(m, 1H, 6′-H), 7.60(s, 1H, 5-H), 7.39-7.35(m, 1H, 4′-H), 7.32(s, 1H, 8-H), 6.63-6.58(m, 1H, 5′-H), 5.96(s, 1H, —OH), 3.97(s,3H, —OCH₃), 3.94(s, 3H, —OCH₃). UV(MeOH): 204.0, 238.0, 321.0 nm. IR(KBr)νₘₐₓ: 3500, 3446, 2960, 1475, 1236, 1375, 1182 cm⁻¹. GC/MS m/z 299(M⁻=1, 100), 298(M³⁰ , 34), 289(11), 291(9). Found: C, 60.26; H, 4.81; N, 18.68. C₁₅H₁₄N₄O₅, requires: C, 60.26; H, 4.81; N, 18.68%.

### 4-(3′-Bromo-4′-hydroxylphenyl)-amino-6,7-dimethoxyquinazoline(HI-P154)

yield 89.90%; m.p.233.0-233.5° C. ¹H NMR(DMSO-d₆): 10.08(s, 1h, —NH), 9.38(s, 1H, —OH), 8.40(s, 1H 2-H), 7.89(d, 1H, J_{2′,6′}=2.7 Hz, 2′-H), 7.75(s, 1H, 5-h), 7.55(dd, 1H, J_{5′,6′}=9.0 Hz, J_{2′,6′}=2.7 Hz, 6′-H), 7.14(s, 1H, 8-H), 6.97(d, 1H, J_{5′,6′}=9.0 Hz, 5′-H), 3.92(s, 3H, —OCH₃), 3.90(s, 3H, —OCH₃). UV(MeOH): 203.0, 222.0, 25.0, 335.0 nm. IR(KBr)νₘₐₓ: 3431(br), 2841, 1624, 1498, 1423, 1244 cm⁻¹. GC/MS m/z 378(M⁺=2, 90.68), 377(M⁺=1, 37.49), 376(M³⁰ , 100.00), 360(MK³⁰ , 3.63), 298(28.86), 282 (6.65). Anal. (C₁₆H₁₄BrN₃O₃,HCl) C, H, N.

### 4-(3′-Hydroxyphenyl)-amino-6,7-dimethoxyquinazoline(HI-P180)

Yield 71.55%; m.p. 256.0-258.0° C. IR(KBr)νₘₐₓ: 3394, 2836, 1626, 1508, 1429, 1251 cm⁻¹. ¹H NMR(DMSO-d₆): 9.41(s, 1H, —NH), 9.36(s, 1H, —OH), 8.46(s, 1H, 2-H), 7.84(s, 1H, 5-H), 7.84-6.50(m, 4H, 2′,4′,5′,6′-H), 7.20(s, 1H, 8-H), 3.96(s, 3H, —OCH³), 3.93(s, 3H —OCH₃). GC/MS m/z: (C₁₆H₁₅N₃O₃.HCl) C, H, N.

### 4-(4′-Hydroxyl-3′-Carboxyphenyl)-amino-6,7-dimethoxyquinazoline (HI-P182)

Yield 79.25%; m.p.>300.0° C. ⁻H NMR(DMSO-d₆)I: δ 10.53(s, 1H, —NH), 8.53(s, 1H, 2-H), 8.10-78.2(m, o3H, 2′,5′,6′, —H), 7.26(s, 1H, 5-H), 6.9(s, 1H, 80H), 4.01(s,3H, —OCH₃), 3.99(s, 3H, —OCH₃). UV(MeOH): 210.0, 239.0, 335.0 nm. IR(KBr)νₘₐₓ 3421, 2839, 1686, 1631, 1508, 1491, 1280 cm⁻¹. GC/MS m/z 341(M³⁰ , 7.91), 323(M⁺—OH, 12.19), 297(M⁺—COOH, 12.35), 296(M⁺—COOH−1.1760), 295(M⁺—COOH−2, 28.65), 206 (11.28).

### 4-(4′-Hydroxyl-3′,5′-dicholophenyl-6,7-dimethoxyquinazoline(HI-P189)

Yield 79.45%; m.p. 293.0-295.0° C. ¹H NMR(DMSO-d₆): 11.32(s, 1H, —NH), 10.34(a, 1H, —OH), 8.87(s, 1H, 2-H), 8.29(s, 1H, 5-H), 7.90(s, 2H, 2′,6′-H), 7.32(s, 1H, 8-H), 4.01(s, 3H, —OCH₃), 3.99(s, 3H, —OCH₃). UV(MeOH): 213.0, 232.0, 250.0, 335.0 nm. IR(KBr)νₘₐₓ: 3479, 2564, 1641, 1579, 1429, 1282, 1147 cm⁻¹. GC/MS m/z 367(M⁺+2, 66.57), 366(M⁺+1, 75.91), 365(M⁺, 100.00), 364(M⁺−1, 94.08), 349(M⁻OH, 11.16. Anal. (C₁₆H₁₃Cl₂N₃O₃) C, H, N. Found: C, 48.93; H. 4.51; N, 10.00. —H₁—Cl₂N₃O₃. HCl requires: C, 48.80; H, 4.31; N, 10.04%.

### 4-(4′-Bromo-6′-hydroxymethylphenyl)-amino-6,7-dimethoxyquinazoline(HI-P190)

Yield 7o3.08%; m.p. 222.0-223.0° C. ¹H NMR(DMSO-d₆): δ 11.30(s, 1H, —OH), 8.22(s, 1H, —NH)O, 7.77.7.23(m, 5H, 5, 8, 2′,3′,5′-H), 4.49(s, 2H, PhCH₂—H), 4.01(s, 3H, —OCH₃), 3.90(s, 3H, —OCH₃). UV(MeOH): 207.0, 250.0, 332.0 nm. IR(KBr)νₘₐₓ: 3446, 2829, 2752, 1652, 1560, 1471, 1365, 1280 cm⁻¹. GC/MS m/z391(M⁻=1, 29.33), 389(M⁻, 29.82), 360(M⁻CH²OH, 50.76), 358(52.39), 311(18.33). 280(43.20), 206(62.80),191(100.00). Anal. (C¹⁷H¹⁶BrN₃O₃HCl) C, H, N.

### 4-(6′-Hydroxymethylphenyl)-amino-6,7-dimethosyquinazoline(HI-P191)

Yield 78.45%; m.p. 215.0-218.0° C. ¹H NMR(DMSO-d₆): δ 11.54(s, 1H, —NH)O, 8.70(s, 1H, 2-H), 8.34(s, 1H, 5-H), 7.62-7.33(m, 4H, 3′,4′,5′,6′-H), 7.39(s, 1H, 8-H), 4.49(s, 2H, PhCH₂OH), 3.99(s, 3H, —OCH₃), 3.98(s, 3H, —OCH₃). UV9MeOH): 209.0, 224.0, 246.0, 335.0 nm. IR(KBr)νₘₐₓ: 3421, 2941, 1675, 2606, '128, 1508, 1437o, 1244cm⁻¹. GC/MS m/z 311(M⁻, 38.07), 310(M⁻−1, 27.04), 28o0 (M⁻CH₂OH, 100.00), 206(17.24), 191(51.34).

### 4-(2′,4′-Dihydroxyphenyl)-amino-6,7-dimethoxyquinazoline (HI-P192)

Yield 86.25%; m.p. 240.0° C.(dec). ¹H NMR(DMSO-d₆): 10.92(s, 1H, —NH), 976(s, 1H, —OH), 9.59(s, 1H, —OH), 8.67(s, 1H, 20H), 81.9(s, 1H, 8-H), 7.36(s, 1H, 50H), 705(d, 1H, J−8.7 Hz, 1′-H), 6.51(s, 1H, 5′-H), 6.31(d, 1H, J−8.7 Hz, 3′-H), 3.98(s,6H, —OCH₃). UV(MeOH): 206.0, 209.0, 223.0, 250.0, 342.0, 486 nm. IR(KBR)νₘₐₓ: 3391, 3139, 2938, 2850, 1633, 1607, 1567, 1509, 1447, 1359, 1220, 1189, 1055 cm⁻¹. GC/MS m/z: 314 (M⁻=1, 13.00), 313 (m⁻, 72.80), 312(m⁺−1, 10.20), 296 (5.24), 206(17.50).

### 4-(2′,3′-Dihydroxyphenyl)-amino-6,7-dimethoxyquinazoline (HI-P192)

Yield 86.25%; m.p 240.0° C.(dec). ¹H NMR(DMSO-d₆): 10.00(s, 1H, —NH), 9.37(s, 1H, —OH), 8.41(s, 1H, 2-H), 7.78(s, 1H, 5-H), 7.49(d, 1H, J_{2′,3′}=2.7 Hz, 2′-H), 7.55(dd, 1H, J_{5′,6′}=9.0 Hz, J_{2′,6′}=2.7 Hz, 6′-H), 7.16(s, 1H, 8-H), 6.97(d, 1H, J_{5′,6′}=9.0 Hz, 5′-h), 3.93(s, 3H, —OCH₃), 3.91(s, 3H, —OCH₃). UV9MeOH): 209.0, 224.0, 249.0, 330.0 nm. IR(KBr)νₘₐₓ: 3448, 2842, 1623, 1506, 1423, 1241 cm⁻¹. GC/MS m/z: 341(M⁺, 100.00), 326(M⁻CH₃, 98.50), 310(M⁺—OCH₃, 12.5), 295(9.0), 189(13.5), 155(13.8). Found: C, 52.35; H, 4.16; Cl, 19.15; N, 11.39. C₁₆H₁₄CIN₃O₃HCl requires: C, 52.32.; H, 4.09; Cl, 19.07; N, 11.44%.

### 4-(2′,4′-Dihydroxyl-1′,3′-diazine-5′)-amino-6,7-dime-thoxyquinazoline (HI-P215)

(Yield 89.23%, m.p.>300.0° C.) ¹H NMR(DMSO-d₆): δ 8.59(s, 1H, 2-H), 7.89(s, 1H, 5-H), 7.60(d, 1H, 6′-H), 7.09(s, 1H, 8-H), 3.78(s, 3H, —OCH₃), 3.76(s, 3H, —OCH₃). UV(MeOH): 222.0, 246.0, 331.0 nm. IR(KBr)νₘₐₓ: 3446, 3212, 3057, 1750, 1682, 1620, 1590, 1511, 1420, 1265 cm⁻¹. GC/MS m/z: 315(M⁻.57.52), 206(46.50), 191(18.21), 127(100.00).

### 4-(3′-Hydroxymethylphenyl)-amino-6,7-dimethoxyquina-zoline(HI-P259)

Yield 74.28%; m.p. 230.0-232.0° C. ¹H NMR(DMSO-d₆): δ 11.29(s, 1H, —NH), 8.83(s, 1H, 2-H)I, 8.28(s, 1H, 5-H), 7.61-7.25(m, 4H, 2′,4′,5′,6′-H), 7.36(s, 1H, 8H)O, 4.57(s, 2H, —CH2OH), 4.02(s, 3H, —OCH₃), 4.00(s, 3H, —OC₃). UV(MeOH): 207.0, 224.0, 251.0, 334.0 nm. IR(KBr)νₘₐₓ: 3500, 3029, 1639, 1589, 1514, 1456, 1284 cm⁻¹. GC/MS m/z: 281(M−+1-CH₂OH, 54.00), 280(M⁻CH2OH, 100.00). Found: C, 58.68; H;, 5.30; N, 12.02. C₁₆H₁₅N₃O₂. HCl requires: C, 58.79; H, 5.19; N, 12.10%.

### 4-[4′-(2″-Hydroxylethylphenyl)]-amino-6,7-dimethoxyqui-nazoline (HI-P265)

Yield 92.30%; m.p. 235.0-240.0° C. ¹H NMR(DMSO-d₆): δ 11.44(s, 1H, —NH), 8.79(s, 1H, 2-H), 8.34(s, 1H, 5-h)I, 7.56(d, 2H, J=8.1 Hz, 2′,6′-H), 7.34(d, 2H, J−8.1 Hz, 3′,5′-H), 7.31(s, 1H, 8H), 4.00(s, 3H, —OCH₃), 3.99(s, 3H, —OCH₃), 3.64(t, 2H,j=6.9 Hz, 1″-H)I, 2.77(t, 2H, J=6.9 Hz, 2″-H). UV(MeOH): 204.0, 226.0, 251.0, 335.0 m. IR(KBr)νₘₐₓ: 3361, 3015, 27o6o7, 1628, 1581, 1514, 1432, 1282 cm⁻¹. GC/MS m/z: 281(17.00), 253(10.00), 207(100.00).

### 4-[2′-(2″-Hydroxylethylphenyl)]-amino-6,7-dimethoxyqui-nazoline(HI P266)

Yield 87.69%; m.p/228.0-230.0° C. ¹H NMR-DMSO-d₆): δ 11.32(s, 1H, —NH), 8.74(s, 14, 2′-H), 8.13(s, 1H, 5-H), 7.46-7.34(m, 4H, 3′,4′,5,6′-H), 7.32(s, 1H, 8H), 4.00(s, 3H, —OCH₃), 3.99(s, eH, —OCH₃), 3.58(t, 2H, J−7.2 Hz, 1″-H), 2.75(t, 2H, J=7.2 Hz, 2″-H). UV(MeOH): 210.0, 226.0, 249.0, 332.0 nm. IR(KBr)νₘₐₓ: 3366, 3226, 3056, 2917o, 2685, 21638, 1571, 1514, 1467, 1277 cm⁻¹. GC/MS m/z: 281(20.00), 253(9.00), 207(100.00).

### 4-(1′-Naphthol-4′)-amino-6,7-edimethoxyquinazoline(HI-P274)

Yield 79.26; m.p. 205.0-208.0° C. ¹H NMR-DMSO-d₆): δ 11.64(s, 1H, —NH), 10.61(s, 1H, —OH), 8.59,(s, 1H, 2-h), 8.41(s, 1H, 5-H), 8.22-6.98(m, 5H, 3′,5′,6′,7′,8′-H), 7.40(s, 1H, 8H), 4.00(s, 3H, —OCH₃), 3.99(s, 3H, —OCH₃). UV9MeOH): 208.0, 215.0, 225.0, 240.0, 330.0 nm. IR(KBr)νₘₐₓ: 3438, 3211, 3061, 2932, 2834, 1633, 1576, 1509, 1437, 1380, 1276, 1215 cm⁻¹. GC/MS m/z: 281(51.00), 253(22.00), 207(88.00). Found: C, 62.26; H, 4.87; N, 10.77. C₂₀H₁₇N₃O₃.HCl requires: C, 62.66; H, 4.70; N, 10.96%.

### 4-(2′-Naphthol-1)-amino-6,7-dimethoxyquinazoline(HI-P275)

Yield 83.17%; m.p 218.0-220.0° C. ¹H NMR(DMSO-d₆): δ 11.33(s, 1H, —NH), 10.22(s, 1H, —OH), 8.62(s, 1H, 2-H), 8.40(s, 1H, 5-H), 7.98-7.31(m, 6H, 3′,4′,5′,6′,7″8′-H), 7.41(s, 1H, 8H), 4.02(s, 3H, —OCH₂), 4.00(s, 3H, —OCH₃),. UV(MeOH): 206.0, 210, 219.0, 225.0, 230.0, 340,0 nm. IR(KBr)νₘₐₓ: 3391, 3165, 3051, 2938, 2840, 1628, 1576, 1504, 1437, 1281, 1215 cm⁻¹. GC/MS m/z: 348(M⁺1, 7.00), 347(M⁻, 100.00), 346(M⁻1.22.00), 331(15.00), 330(12.00), 281(23.00), 253(12.00), 207(49.00). Found: C, 62.91; H, 4.76; N, 10.75. C₂₀H₁N₃O₃.HCl requires: C, 62.66; H, 4,70; N, 10.96%.

### 4-[3′-(1″-Hydroxyethyl)]-amino-6,7-dimethoxyquinazoline (HI-P276)

Yield 79.21%; m.p. 215.0-218.0° C. ¹H NMR(DMSO-d₆): δ 11.40(s, 1H, —NH), 8.81(s, 1H, 20H), 8.31(s, 1H, 5-H)O, 7.60-7.26(m, 4H, 2′,4′,5′,6′-H), 7.41(s, 1H, 8H), 4.65(q, 1H, J=6.6 Hz, —CH(OH)CH₃), 4.00(s, 3H, —OCH₃), 3.98(s, 3H, —OCH₃), 1.350(d, 3H, J=6.6 Hz, —CH₃). UV9MeOH): 204.0, 216.0, 220.0, 224.0, 250.00, 348.0 nm. IR(KBr)νₘₐₓ: 3407, 3030, 2977, 2840, 1643, 1591 1514, 1463, 1370, 1282, 1230 cm⁻¹. GC/MS m/z: 325(M⁻+1, 67.00), 324(M⁻, 100.00), 323(M⁻1.22.00), 308(17.00), 307(56.00), 306(21.00), 281(2.00), 280(8.00), 264(6.00).

### 4-(4′-Hydroxy-3′,5′-diphenylphenyl)-amino-6,7-dime-hoxyquinazoline (HI-P277)

Yield 76.11%; m.p. 255.0-257.0° C. ¹H NMR_DMSO-d₆): δ 11.50(s, 1H, —NH), 8.80(d, d, 2H, 2′,6′-H), 8.58(s, 1H, 5-H), 7.60-7.30(m, 10H, 3′,5′, Ph-H), 7.39(s, 1H, 8H), 4.00(s, 3H, —OCH₃), 3.97(s, 3H, —OCH₃), 1.350(d, eH, J=6.6 Hz, —CH₃). UV(MeOH): 210.0, 214.0, 229.0, 239.0, 345.0, 248.0, 352.0 nm. IR(KBr)νₘₐₓ: 3520, 3218, 3023, 2935, 1630, 1562, 1518, 1457, 1281, 1234 cm. GC/MS m/z: 281(35.00), 267(6.00), 253(10.00), 207(100.00).

### 4-(4′-Hydroxyl-2′-chlorophenyl)-amino-6,7-dimethoxy-quinazoline(HI-P2878)

Yield 81.44%; m.p.245.0-247.0° C. ¹H NMR(DMSO-d₆): δ 11.39(s,1H,—NH)O, 10.30(s, 1H, —OH), 8.75(s, 1H, 2-H), 8.24(s, 1H, 5-H), 7.38-6.85(m, 3H, 3′,5′,6′-H), 7.37(s, 1H, 8H), 3.98(s, 3H, —OCH₃), 3.96(s, H3, —OCH₃). UV(MeOH): 222.0, 234.0, 239.0, 245.0, 254.0 348.0 nm. (R(KBr)νₘₐₓ: 3448, 3242, 3144, 3025, 2917, 2834, 1638, 1591, 1514, 1437, 1365, 1277, 1209 cm⁻¹. GC/MS c/z: 332(M⁻+1, 5.00), 331(M⁻, 17.00), 330(M⁻−1, 5.00), 297(17.00), 296(100.00), 281(18.00), 280o(29.00), 253(9.00).

### 4-(2′-Hydroxy-naphthyl-3′)-amino-6,7-dimethoxyquinazolin(HI-P292)

Yield 87.41%; m.p. 277.0-279.0° C. ¹H NMR(DMSO-d₆): δ 11.38(s, 1H, —NH)O, 10.35(s, 1H, —OH), 8.73(s, 1H, 2-H), 8.25(s, 1H, 5-H), 7.93-7.30(m, 6H, 1′,4′,5′,6′,7′, 8′-H), 7.37(s, 1H, 8H)O, 4.00(s, 6H, —OCH₃). UV(MeOH): 204.0, 221.0, 224.0, 230.0, 256.0, 344.0 nm. IR(KBr)νₘₐₓ: 3479, 3386, 3036, 2901, 1632, 1581, 1504, 1437, 1281 cm⁻¹. GC/MS m/z: 281(41.00), 253(11.00), 207(100.00). Found: C, 62.87; H;, 4.83; N, 1o0.78. C₂₀H₁N₃O₃. HCl requires: C, 62.66; H, 4.70, N, 10.96%.

### 4-(1′-Hydroxy-naphthyl-5′)-amino-6,70-dimethoxyquina-zoline(HI-P293)

Yield 87.21%; m.p. 204.0-205.0° C. ¹H NMR(DMSO-d₆): δ 11.73(s, 1H, —NH), 10.43(s, 1H, —OH), 8.65(s, 1H, 2-H, 8.38(s, 1H, 5-H), 8.21-6.95(m, 6H, 2′,3′,4′,6′,7′,8′-H), 7.33(s, 1H, 8H)O, 4.00(s, 6H, —OCH₃). UV9MeOH): 204.0, 214.0, 224.0, 229.0, 235.0 348 nm. IR(KBrνₘₐₓ: 3449, 3335, 3102, 2927o, 1633, 1571, 1509, 1437, 1287 cm⁻¹. Found: C, 62.23; H, 4.96; N, 10.89. C₂₀H₁₇N₃O₃.HCl requires. C, 62.66; H, 4.70; N, 10.96%.

### 4-(4′-Hydroxy-3,5-diiodophenyl)-amino-6,7-dimethoxy-quinazoline(HI-P294)

Yield 77.47&; m.p. 259.0-260.0° C. ¹H NMR(DMSO-d₆): δ 11.13(s, 1H, NH), 9.73(s, 1H, —OH), 8.87(s, 1H, 2-H), 8.16(s, 1H, 5-H), 8.09(s, 2H, 1′,6′-H), 7.28(s, 1H, 8H), 3.98(s, 6H, —OCH₃),. UV(MeOH)λₘₐₓ): 217.0, 227.0, 252.00 nm. IR(KBrνₘₐₓ: 3457, 3201, 2934, 2832, 2566, 1629, 1562, 1521, 1439, 1275, 1075 cm⁻¹. GC/MS m/z: GC/MS m/z 422(M⁻I.33.53), 405(7.50), 281(86.67), 221 (51.80), 207(91.30). Found: C, 32.60; H, 2.50; N, 6.92. C₁₆H₁₃I₂N₃O₃.HCl requires: C. 32/82.′ J. 2.39; N, 7.18%.

### 4-(4′-Hydroxy-3′-iodophenyl)-amino-6,7-dimethoxyquinazoline(HI-P299)

Yield 71.59%; m.p. 248.0-250.0° C. ¹H NMR(DMSO-d₆): δ 11.32(d, 1H, NHO), 10.62(s, 1H, —OH, 8.79(s, 1H, 2-H), 8.26(s, 1H, 5-H), 7.98-6.98(m, 3H, 2′,3′,6′-H), 7.32(s, 1H[, 8H), 3.98(s, 3H, —OCH₃), 3.97(s, 3H, —OCH₃). UV(MeOH)λₘₐₓ (ε):. 217.0, 227.0, 252.0 nm. IR(KBr)νₘₐₓ: 3411, 2975, 2730, 2366, 1634, 1573, 1501, 1429, 1229, 1075 cm⁻¹. GC/MS m/z: 406(M⁻1.3.33), 405(M⁻2, 7.50), 281(M⁺−1-I, 26.67), 253(11.80), 207(100.00). Found: C, 41.96; H, 3.40; N, 8.98. C₁₆H₁₄IN₃O₃.HCl requires: C, 41.83; H, 3.26; N, 9.15%.

TABLE 5
Fluoroquinazoline Derivatives

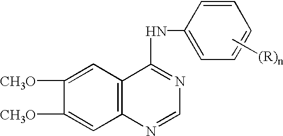

| (HI-P352) | | (HI-P353) | |
|---|---|---|---|
| No | R | Formular | MW |
| HI-P144 | 2-F, 3-F, 5-F, 6-F, 4-Br | C₁₆H₁₀BrF₄N₃O₂ | 432 |
| HI-P214 | 2-F, 4-Br | C₁₆H₁₃BrFN₃O₂ | 378 |
| HI-P218 | 3-CF₃ | C₁₇H₁₄F₃N₃O₂ | 349 |
| HI-P219 | 4-OCF₃ | C₁₇H₁₄F₃N₃O₃ | 365 |
| HI-P221 | 4-F | C₁₆H₁₄FN₃O₂ | 299 |
| HI-P223 | 4-CF₃ | C₁₇H₁₄F₃N₃O₂ | 349 |
| HI-P224 | 3-F | C₁₆H₁₄FN₃O₂ | 299 |
| HI-P228 | 2-CF₃ | C₁₇H₁₄F₃N₃O₂ | 349 |
| HI-P232 | 4-SO₂F | C₁₆H₁₄FN₃O₄S | 363 |
| HI-P264 | 2-F | C₁₆H₁₄FN₃O₂ | 299 |
| HI-P352 | * | C₂₅H₂₀F₆N₄O₂ | 522 |
| HI-P353 | * | C₂₅H₂₀F₆N₄O₂ | 522 |
| HI-P364 | 3-OCF₃ | C₁₇H₁₄F₃N₃O₃ | 365 |
| HI-P365 | 2-OCF₃ | C₁₇H₁₄F₃N₃O₃ | 365 |
| HI-P366 | 3-CF₃, 5-CF₃, | C₁₈H₁₃F₆N₃O₂ | 417 |
| HI-P367 | 2-CF₃, 5-CF₃, | C₁₈H₁₃F₆N₃O₂ | 417 |
| HI-P369 | 3-F, 4-OH | C₁₆H₁₄FN₃O₃ | 315 |
| HI-P408 | 3-F, 5-F, 4-OH | C₁₆H₁₃F₂N₃O₃ | 333 |

HI-P352

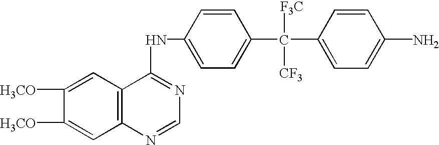

HI-P353

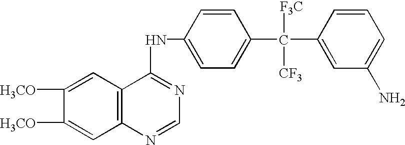

### Example 6

| No | Name | Structure | Formula | MW |
|---|---|---|---|---|
| 1 | P-144 | 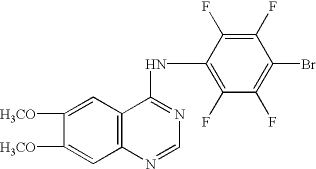 | C₁₆H₁₀BrF₄N₃O₂ | 432 |
| 2 | P-214 | 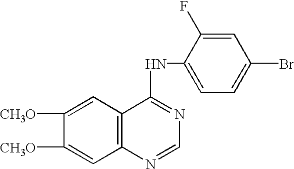 | C₁₆H₁₃BrFN₃O₂ | 378 |
| 3 | P-218 | 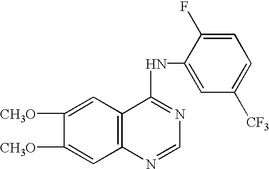 | C₁₇H₁₃F₄N₃O₂ | 367 |
| 4 | P-219 | 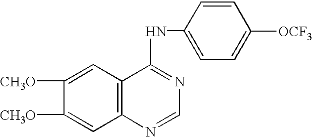 | C₁₇H₁₄F₃N₃O₃ | 365 |
| 5 | P-221 | 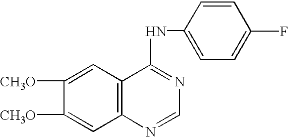 | C₁₆H₁₄FN₃O₂ | 299 |
| 6 | P-223 | 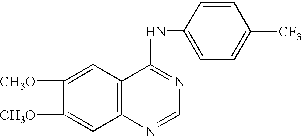 | C₁₇H₁₄F₃N₃O₂ | 349 |
| 7 | P-224 | 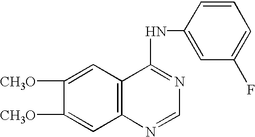 | C₁₆H₁₄FN₃O₂ | 299 |
| 8 | P-228 | 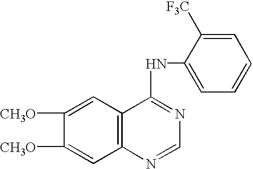 | C₁₇H₁₄F₃N₃O₂ | 349 |
| 9 | P-232 | 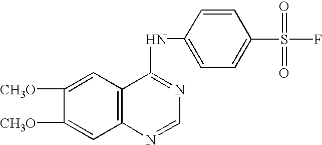 | C₁₆H₁₄F₂SN₃O₄ | 363 |
| 10 | P-264 | 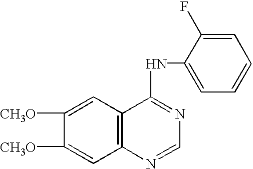 | C₁₆H₁₄FN₃O₂ | 299 |
| 11 | P-352 | 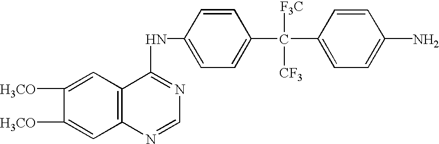 | C₂₅H₂₀F₆N₄O₂ | 522 |
| 12 | P-353 | 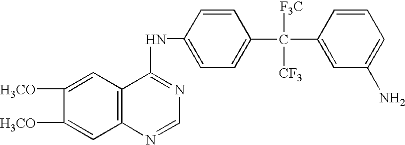 | C₂₅H₂₀F₆N₄O₂ | 522 |
| 13 | P-364 | 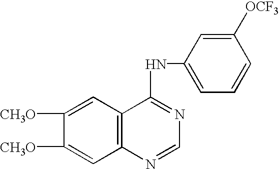 | C₁₇H₁₄F₃N₃O₃ | 365 |
| 14 | P-365 | 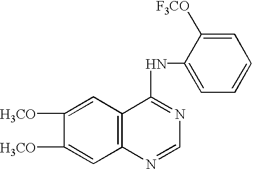 | C₁₇H₁₄F₃N₃O₃ | 365 |
| 15 | P-366 | 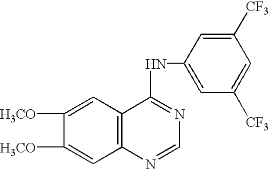 | C₁₈H₁₃F₆N₃O₂ | 417 |
| 16 | P-367 | 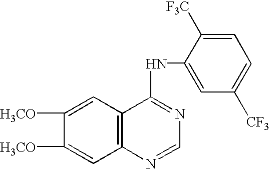 | C₁₈H₁₃F₆N₃O₂ | 417 |
| 17 | P-369 | 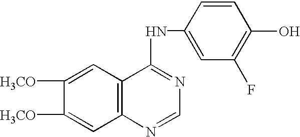 | C₁₆H₁₄FN₃O₃ | 315 |
| 18 | P-408 | 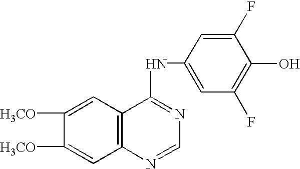 | C₁₆H₁₃F₂N₃O₃ | 333 |

### 4-(2′,3′,5′,6′-Terrafluoro-4 bromophenyl)-amino-6,7-dime-thoxyquinazoline (HI-P144)

The yield 78.24%: m.p. 180.0-182.0 0° C. ¹H NMR (DMS O-d): Λ 7.78(s. 1H. 2-H), 7.53(s. 1H, 5-H), 6.79(s. 1H, 8-H), 3.81(s.3H, —OCH₃), 3.3.79(s.3 H, —OCH₃). Found: C, 41.12; H, 2.41: N, 9.89, C₁₀H₁₀BrFN₃O₂.HCl. requires: C, 41.11; H, 2.36; N, 9.93%.

### 4-(2′-Fluoro-4′-bromophenyl)-amino-6,7-dimethoxyquina-zoline (HI-P214)

The yield 77.21%; m.p. 247.0-252.0 0° C. ¹H NMR(DMSO-d₆): Λ 8.57(s. 1H. 2-H), 7.91(s. 1H, 5-H), 7.57 (d. 1H, 3′-H), 7.34(m. 2H. 5′,6′-H). 7.07(s. 1H, 8-H), 3.78(s. 3H. —OCH₃), 3.77(s. 3H. —OCH₃). UV(MeOH):.204.0, 215.0, 250.0, 330.0 nm. IR(KBr) νₘₐₓ: 3431, 2629, 1633, 1580, 1511, 1420, 1278cm⁻¹. GC/MS m/z 379(M⁺+1,34.39), 378(M⁻,31.33). 377(M⁻−1,39.08), 360(62.05), 359 (31.58), 358(62.57), 357(19.81), 299(19.31), 298(100.00), 282(17.88), 240(28.76).

### 4-(3′-Trifluoromethylphenyl)-amino-6,7-dimethoxyquinazo-line (HI-P218)

The yield 85.61%: m.p. 242.0-245.0 0° C. ¹H NMR(DMSO-d₆): Λ 11.09(s. 1H. —NH). 8.67(s. 1H. 2-H), 8.03(s, 1H, 5-H), 7.92-7.43(m, 4H, 2′,4′5′,6′-H). 7.10(s. 1H. 8-H). 3.81(s, 3H, —OCH₃), 3.79(s,3H, —OCH₃). UV(MeOH):. 206.0. 276.0, 349.0 nm. IR νₘₐₓ (KBr): 3372, 3257, 2935, 1626, 1512, 1380, 1225 cm⁻¹. GC/MS m/z 350(M⁺+1, 10.5), 249(M⁻.85.5). 173(M⁻−1,100.0), 332(10.5), 290 (8.8).

### 4-(4′-Trifluoromethoxylphenyl)-amino-6,7-dimethoxyqui-nazoline (HI-P219)

The yield 83.14%; m.p. 228.0-230.0 0° C. ¹H NMR(DMSO-d₆): Λ 11.39(s, 1H, —HN), 8.63(s, 1H, 2-H), 8.18(s, 1H, 5- H), 7.63(t, 2H, 3′,5′-H). 7.27(t, 2H, 2′. 6′-H). 7.15(s. 1H, 8-H), 3.81(s, 3H, —OCH₃), 3.78(s, 3H, —OCH₃). UV(MeOH):. 209.0, 216.0, 251.0, 332.0 nm. IR(KBr)νₘₐₓ: 3207, 2839, 2762, 1633, 1508, 1480, 1276 cm⁻¹. GC/MS m/z 366(M⁺+1, 12.50). 365(M⁻, 75.00). 364(M⁻−1, 100.00), 348(17.50), 319(19), 306(8.00). 207(15.00).

### 4-(4′-Fluorophenyl)-amino-6,7-dimethoxyquinazoline(HI-P221)

The yield 84.25%:. ¹H NMR(DMSO-d₆): Λ 11.19(s. 1H, —HN). 8.60(s. 1H, 2-H). 8.08(s. 1H, 5- H)). 7.50(t, 2H, 3′-H), 7.13(s. 1H, 8-H), 7.12(t. 2H, 2′,6′-H). 3.79(s. 3H. —OCH₃), 3.78(s, 3H, —OCH₃). UV (MeOH):. 225.0, 251.0, 333.0 nm. IR (KBr)νₘₐₓ: 3205, 3007, 2837, 1633, 1580, 1508, 1470, 1220 cm⁻¹. GC/MS m/z 300(M⁺+1, 10.76), 299(m⁻, 76.92), 398(M⁻−1 , 100.00), 282(20.00). 253(13.08), 207(3.80). Found: C, 57.17; H, 4.37; N, 12.47, C₁₆H₁₄FN₃O₂.HCl requires C, 57,31: H, 4.48; N, 12.54%.

### 4-(4′-Trifluoromethylphenyl)-amino-6,7-dimethoxyquinazoline (HI-P223)

The yield 91.70%: m.p. 243.0-245.0 0° C. ¹H NMR(DMSO-d₆): Λ 11.47(s. 1H. —NH), 8.67(s. 1H, 2-H), 8.23(s. 1H, 5-H), 7.79(d. 2H, J=8.4 Hz. 3′5′-H). 7.61(d. 2H. J=8.4 Hz. 2′6′-H), 7.17(s. 1H, 8-H), 3.82(s. 3H. —OCH₃), 3.78(s. 3H, —OCH₃). GC/MS m/z 350(M⁻+1, 11.00). 349(M⁻, 65.00), 348(M⁻−1, 100.00), 332(18.50), 303(10.00), 207(18.50). Found: C, 53.01; H. 3.94; N, 10.88. C₁—H₁₄F₃N₃O₂HCl requires C. 52.98; H. 3.90: N, 10.91%.

### 4-(4′-Flurophenyl)-amino-6,7-dimethoxyquinazoline(HI-P224)

The yield 88.69%; m.p. 254.0-255.0 0° C. ¹H NMr(DMSO-d₆): δ 11.16(s, 1H, —HN), 8.67(s, 1H, 2-H), 8.09(s, 1H, 5-H), 7.13(s, 1H, 8-H), 7.51-6/94(m, 4H, 2′,3′,5′,6′-H)O, 3.80(s, 3H, —OCH₃), 3.79(s, 3H, —OCH₃). UV(MeOH): 206.0, 226.0, 251.0, 333.0, 343 nm. IR(KBr)νₘₐₓ: 3437, 3211, 2619, 1637, 1580, 1500, 1448, 1281 cm⁻¹. GC/MS m/z(300(M⁺+1, 8.00), 299(M⁻, 68.00), 398(M⁻1, 100.00), 282(21.60), 253(25.00), 207 (80.00),. Found: C, 57.25; H, 4.58; N, 12.42. C₁₆H₁₄FN₃O₂.Hcl requires C, 57.31; H, 4.48; N, 12.54%.

### 4-(2′-Trifluoromethylphenyl)-amino-6,7-dimethoxyquinazoline(HI-P228)

The yield 83.57%; m.p. 242.0-245.0 0° C. ¹H NMR(DMSO-d₆): δ 11.58(s, 1H, —HN), o8.76(s, 1H, 2-H), 8.25(s, 1H, 5-H), 7.95-7.62(m, 4H, 3′,4′,5′,6′-H), 7.38(s, 1H, 8-H), 4.01(s, 3H, —OCH₃), 3.00(s, 3H, —OCH₃). GC/MS m/z 350(M⁻+1, 8.50), 349(M⁻, 32.00), 348(M⁺−1.31.50), 328(18.50), 207(5.0)I, 280(M⁺—CF₃, 100.00), 264(18.50), 207(32.50). Found: C, 52.71; H, 4.26; N, 10.91%.

### 4-[4′-benzenesulfanilyl fluoride]-amino-6,7-dimethoxyquinazoline (HI-P232)

Yield 84.02%; m.p. 228.0-230.0° C. ¹H NMR9DMSO-d₆): δ 11.62(s, 1H, —HN), 8.78(s, 1H, 2-H), 8.29(s, 1H, 5-H), 8.12-8.02(m, 4H, 2″,3″,5″,6″-H), 7.21(s, 1H, 8-H), 3.86(s, 3H, —OCH₃), 3.81(s, 3H, —OCH₃). UV(MeOH): 208.0, 215.0, 253.0, 278.0, 338.0 nm. IR(KBr)νₘₐₓ: 3440, 3277, 2571, 1635, 1580, 1516, 1435, 1209 cm⁻¹. GC/MS m/z: 281(43.00), 253(12.00), 207(100.00). Found: C, 48.13; H, 3.73; N, 10.53. C₁₆H₁₄FN₃O₄S.HCl requires: C, 48.12; H, 3.76; N, 10.53%.

### 4-(2′-Fluorophenyl)-amino-6,7-dimethoxyquinazoline(HI-P264)

Yield 73.58%; m.p. 233.0-235.0 0° C. ¹H NMR(DMSO-d₆): δ 11.69(d, 1H, —NH), 8.82(s, 1H, 2-H), 8.37(s, 1H,k 50H), 7.59-7.32(m, 4H 3′,4′5′,6′-H), 7.41(s, 1H, 8H)O, 4.02(s, 3H, —OCH₃), 4.01(s, 3H, —OCH₃). UV(MeOH): 204.0, 226.0, 248.0, 330.0 nm. IR(KBrνₘₐₓ: 3454, 3032, 2638, 1630, 1589, 1514, 1430, 1291 cm⁻¹. GC/MS m/z 300(M⁺=1, 7.00), 299(M⁻.38.00), 298(M⁻−1.22.00), 280(M⁻F, 100.00), 264(15.00), 207(35.00). Found: C, 57.12; H, 4.57; N, 12.45. C₁₆H₁₄FN₃O₂.HCl requires: C, 57.31; H, 4.48; N, 12.54%.

### 4-{4′-[2″-(4″′-Aminophenyl)-hexafluoropropyl]phenyl}-amino-6,7-dimethoxyquinazoline(HI-P352)

Yield, 80.41%, m.p. 280.0-282.0° C. ¹H NMR(DMSO-d₆): δ 11.87(s, 1H, —NH), 8.91(s, 1H, 2-H)I, 8.55-7.18(m, 10H, 5, 8, 2′,3′,5′,6′,2″′,3″′,5″′,6″′-H), 4.05(s, 3H, —OCH₃), 4.00(s, 3H, —OCH₃). ¹⁹F NMR(DMSO-d₆): 128.76. Found: C, 50.33; H, 3.87; N, 9.57. C₂₅H₂₀F₆N₄O₂.2HCl requires: C, 50.50; H, 3.70; N, 9.42%

### 4-{3′-[2″-(3″′-Aminophenyl)-hexafluoropropyl]phenyl}-amino-6,7-dimethoxyquinazoline(HI-P353)

Yield, 83.11%,. m.p. 292.0-284.0° C. ¹H NMR(DMSO-d₆): Λ 11.68(s. 1H. —NH). 8.81(s. 1H. 2-H). 8.44-7.09(m. 10H. 5, 8, 2′,4′,5′,6′,2″′,4″′,5″′,6″′-H). 4.00(s. 3H. —OCH₃). 3.97(s. 3H. —OCH₃). ¹⁹F NMR(DMSO-d₆): 129.21. Found: C, 53.96: H,3.93; N,9.77. C₂₅H₂₀F₆N₄O₂.HCl requires: C. 53.76: H.3.76: N. 10.03%

### 4-(3′-Trifluoromethoxylphenyl)-amino-6,7-dimethoxyquinazoline (HI-P364)

Yield. 83.25%. m.p. 233.0-235.0° C. ¹H NMR(DMSO-d₆): Λ 11.65(s, 1H. —NH), 8.88(s. 1H1 2-H), 8.41(s. 1H, 5-H), 7.86-7.29(m, 4H, 2′,4′,5′,6′-H). 7.36(s. 1H, 8-H), 4.02(s. 3H, —OCH₃). 3.98(s. 3H, —OCH₃). ¹⁹F NMR(DMSO-d₆): 135.37. GC/MS m/z: 366(M⁺+1, 11.0), 365(M³⁰ , 67.0), 364(M⁺−1, 100.0). Found: C, 50.93; H,3.75; N,10.61. C₁₇H₁₄F₃N₃O₃.HCl requires: C, 50.97; H.3.74; N, 10.47%.

### 4-(2′-Trifluoromethoxylphenyl)-amino-6,7-dimethoxyquinazoline (HI-P365)

Yield. 77.85%. m.p. 235.0-237.0° C. ¹H NMR(DMSO-d₆): Λ 11.68(s. 1H, —NH), 8.80(s. 1H. 2-H). 8.32(s. 1H, 5-H), 7.64-7.53(m, 4H, 3′,4′,5′,6′-H). 7.40(s. 1H, 8-H), 3.99(s, 6H, —OCH₃). ¹⁹F NMR(DMSO-d₆): 135.71. GC/MS m/z: 366(M⁻+1, 2.0),365(M³⁰ , 15.0), 364(M⁺−1, 4.0), 281(21.0), 280(M⁻—OCF₃ 100). Found: C, 50.83; H.3.79; N,10.52. C₁H₁₄F₃N₃O₃.HCl requires: C, 50.87; H,3.74; N, 10.47%.

### 4-(3′,5′-Ditrifluoromethylphenyl)-amno-6,7-dimethoxyquinazoline (HI-P366)

Yield. 82.88% m.p. 270.0-272.0° C. ¹H NMR(DMSO-d₆): Λ 11.87(s. 1H, —NH), 8.97(s. 1H, 2-H), 8.60)s. 2H, 2′,6′-H). 8.43(s. 1H, 5-H), 7.98(s. 1H, 4′-H), 7.35(s. 1H, 8-H), 4.03(s. 3H, —OCH₃). 3.99(s. 3H, —OCH₃). ¹⁹F NMR (DMSO-d₆): XX GC/MS m/z: 418(M⁻+1. 19.0), 417(M⁻, 100.0), 416(M⁻−1, 73.0), 398(M⁻—F, 16.0), 398(M⁻—F, 16.0), 348(M⁻—CF. 16.0). Found: C, 47.78; H, 3.20; N, 9.26. C₁₈H₁₃F₆N₃O₂.HCl requires: C, 47.68; H, 3.09; N, 9.27%.

### 4-(4′Hydroxyl-3′-fluorophenyl)-amino-6,7-dimethoxyquinazoline (HI-P369)

Yield. 84.28%. m.p. 268.0-270.0° C. ¹H NMR(DMSO-d₆: Λ 11.36(s. 1H, —NH). 10.13(s, 1H, —OH). 8.80(s. 1H, 2-H), 8.30(s. 1H, 5-H), 7.60-7.02(m. 3H. 2′,5′,6′-H). 7.31(s. 1H, 8-H). 3.99(s. 3H, —OCH₃), 3.97(s. 3H, —OCH₃). ¹⁹F NMR(DMSO-d₆): Λ 57.38. Found: C, 54.90: H, 4.28;N, 11.91. C₁₆H₁₄FN₃O₃.HCl requires C, 54.70; H, 4.27; N, 11.97%.

### 4-(4′-Hydroxyl-3′,5′-difluorophenyl)-amino-6,7-dimethoxy-quinazoline (HI-P408)

Yield. 83.15%, m.p.228.0-230.0 0° C. ¹H NMR(DMSO-d₆): Λ 11.46(s. 1H, —NH), 10.39(s. 1H, 2-H), 8.36(s. 1H, 5-H). 7.56, 7.54 (s. s. 2H. 2′,6′-H), 7.33(s. 1H. 8-H), 4.00)s. 3H, —OCH₃), 3.98(s. 3H, —OCH₃). ¹⁹F NMR(DMSO-d₆: Λ 60.25, 60.22. Found: C, 52.04; H, 4.17; N,11.10. C₁₆H₁₃F₂N₃O₃.HCl. requires C, 52.03; H, 3.79; N,11.38%.

### Example 7

### Solubility Profile of WHI-P131

The solubility of WHI-P131 free base was measured in water, propylene glycol, polyethylene glycols (PEGs), ethanol, and triglycerides. The results are summarized in Table 6. The solubility of WHI-P131 is very poor in water. It was about 35 times more soluble in C₈-C₁₀ medium chain triglyceride (Captex 300) than in water. It was much more soluble in ethanol and hydrophilic cosolvents such as propylene glycol and PEGs. WHI-P131 free base was most soluble in polyethylene glycols of greater than 10%, followed by propylene glycol (1.95%) and ethanol (1.86%).

Parallel solubility measurements were also carried out using WHI-P131 chloride salt. Table 1 shows that a 50 fold increase in water solubility was achieved when the free base form was converted into a chloride form. In contrast, the solubility of WHI-P131 chloride decreased drastically in all other liquids. As can be seen it Table 6, its solubility in Captex 300, ethanol, propylene glycol, PEG300, and PEG200 decreased by a factor of about 10 to 70 compared to the compound free base. These results show that the improvement of the solubility in water of the compound salt form was offset by a much larger decrease of its solubility in other liquids. This fact underscores the importance of knowing the solubility profile of both the free base and salt forms of an ionizable compound when making choices of its delivery vehicles.

TABLE 6
Solubility of WHI-P131 (mg/ml) free base
versus WHI-P131 chloride salt in various liquids

| | WHI-P131 | WHI-P131 |
|---|---|---|
| Liquid | Free base | Chloride salt |
| Water | 0.025 ± 0.07 | 1.24 ± 0.09 |
| Captex 300 | 0.88 ± 0.08 | 0.012 ± 0.002 |
| PEG300 | >185 | 10.10 ± 3.60 |
| PEG200 | >100 | 25.12 ± 0.72 |
| Propylene glycol | 19.5 ± 0.7 | 1.61 ± 0.09 |
| Ethanol | 18.6 ± 0.5 | 0.631 ± 0.007 |

### Example 8

### Co-Solvent Vehicles

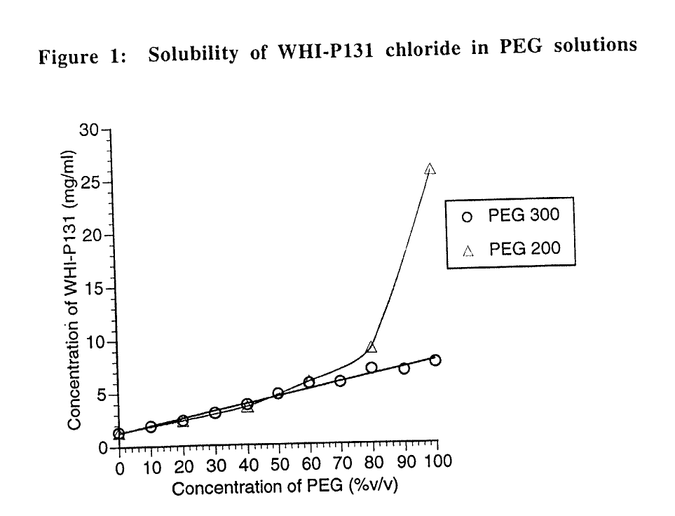

To further determine the effect of cosolvents on the solubility behavior of WHI-P131, the solubility of WHI-P131 chloride salt was carried out in binary mixtures of water-cosolvents. Solvents including ethanol, propylene glycol, and PEGs are used in several injectable formulations, and were considered here as possible vehicles for WHI-P131. FIG. 1 shows that at PEG concentrations below 70% in water, the solubility versus PEG concentration curves were practically superimposable for PEG300 and PEG200. However, at PEG concentrations greater than 70%, there was a large difference between the solubility behavior of WHI-P131 in water-PEG300 and water-PEG200 mixtures. For PEG300, the solubility continued to increase linearly with increasing PEG concentration, whereas for PEG200, a large increase in slopes occurred near 100% PEG200. Since the solubility-PEG300 concentration curve is linear over the entire range of water-PEG300 mixtures, WHI-P131 solubilized in these mixtures at concentrations below its saturation point can be used as vehicles for this compound, since their dilution will not result in drug precipitation. In contrast, if one were to dilute by water a 2% WHI-P131 in PEG200, WHI-P131 concentration would fall above the solubility limit and precipitate out. Therefore, PEG300 is more appropriate for use as a cosolvent vehicle in the formulations of WHI-P131.

### Example 9

### Micellar Solutions

Micellar solutions containing PEGylated phosphatidylethanolamines were exceptionally effective in enhancing the solubilization of WHI-P131. Table 7 shows the compositions of several mixed micellar solutions containing various amounts of WHI-P131. Micellar solutions using purified soya lecithin (Phospholipon 90G) were feasible when an equal or higher amount of a nonionic surfactant (such as Cremophor EL for example) was also present. With PEGylated phospholipids, the presence of Cremophor EL was not necessary to form micellar solutions. In addition, an anionic PEGylated phosphatidylethanolamine seemed to be a better solubilizer for WHI-P131 than Phospholipon (mostly phosphotidylcholine). The enhanced solubilization observed with micellar solutions was apparently due to the charge interaction between the cationic WHI-P131 and anionic PEGylated phosphatidylethanolamine. In Table 7, compositions MM3 and MM4 where PEG2000-DPPE and PEG5000-DPPE were present show the highest solubilization (highest drug to surfactant ratio). It was also found that the presence of Pluronic F-68 aided in preventing drug recrystallization.

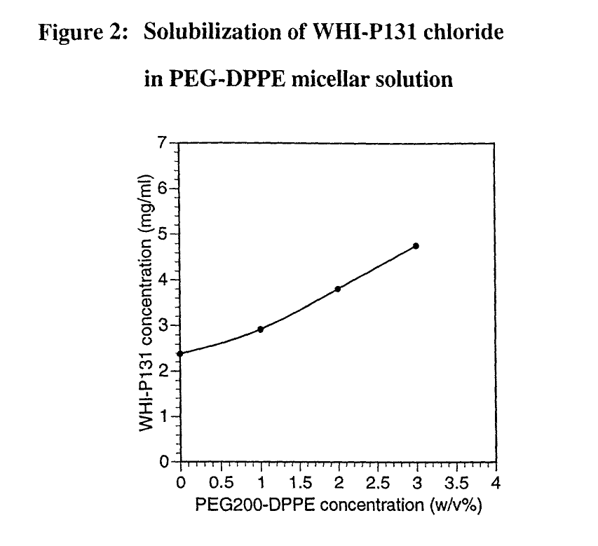

To determine the solubilization enhancement by different types of surfactants in a more quantitative manner, solubility-surfactant concentration curves were plotted. FIG. 2 depicts the amount of solubilized WHI-P131 chloride in a solution containing 20% of PEG300, and an increasing amount of PEG2000-DPPE. This figure indicates that, in the absence of surfactant, the solubility of WHI-P131 chloride salt in 20% PEG300 was 2.38 mg/ml. At low surfactant concentrations (below the CMC), the drug solubilization seems to remain unchanged, then increases linearily with surfactant concentration at higher PEG2000-DPPE concentration. The same solubilization characteristics were observed with other micellar solutions. In Table 8, the slopes of the linear portions of the plot for a series of nonionic surfactants and cosolvents were used to calculate the solubilization enhancement per unit surfactant or cosolvent concentration.

The solubilization enhancement, as represented by the amount of solubilized WHI-P131 (in milligram) per gram of surfactant, are shown in Table 8 to vary with the type of surfactants used. For the three PEGylated phosphatidylethanolamines, the solubilization enhancement depended on the hydrophobic chain length and polyoxyethylene number of the PEGylated phospholipids. PEG2000-DPPE and PEG5000-DPPE seemed to be the most effective solubilizers for WHI-P131 of the three PEGylated phosphatidylethanolamines investigated. Also shown in Table 8 for comparison purposes are the solubilization enhancements produced by the use of cosolvents. It can be seen that PEGylated surfactants were about 6 to 16 times more effective than cosolvents in producing solubilization enhancement of WHI-P131 chloride salt.

TABLE 7
WHI-P131 in mixed micelles

% Concentration

| COMPONENT | MM1 | MM2 | MM3 | MM4 |
|---|---|---|---|---|
| WHI-P131 | 0.18 | 0.26 | 0.43 | 0.37 |
| Phospholipon 90G | 0.0 | 1.28 | 0.0 | 0.0 |
| PEG2000-DPPE (a) | 0.0 | 0.0 | 1.84 | 0.0 |
| PEG5000-DPPE (b) | 1.16 | 0.0 | 0.0 | 2.51 |
| Pluronic F-68 | 0.29 | 0.32 | 0.46 | 0.55 |
| Cremophor EL | 1.45 | 1.6 | 2.29 | 0.0 |
| Propylene glycol | 11.64 | 12.8 | 25.6 | 12.9 |
| Water | 85.3 | 83.7 | 69.4 | 83.7 |
| Surfactants/Drug Ratio | 16.1 | 12.1 | 10.7 | 8.3 |

(a) PEG2000 dipalmitoyl phosphatidylethanolamine
(b) PEG5000 dipalmitoyl phosphatidylethanolamine

TABLE 8
Solubilization enhancement of WHI-P131
in micellar solutions and cosolvent vehicles

| | | Solubilized WHI-P131 (mg) |
|---|---|---|
| Enhancer | Vehicle type | per gram of surfactant or solvent |
| PEG2000-PE (16:0) | Micellar solution | 92.0 |
| PEG2000-PE (14:0) | Micellar solution | 76.6 |
| PEG5000-PE (18:0) | Micellar solution | 37.5 |
| Pluronic F-68 | Micellar solution | 6.1 |
| PEG400 | Cosolvent | 6.2 |
| PEG300 | Cosolvent | 5.8 |
| PEG200 | Cosolvent | 5.3 |

### Example 10

### Microemulsions

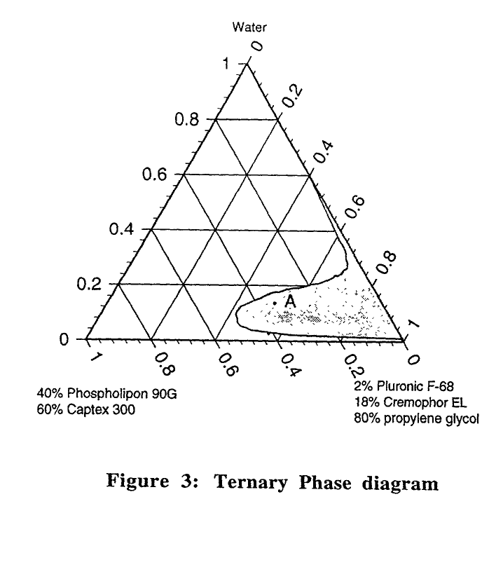

A series of ternary phase diagrams were constructed at room temperature, and several microemulsions within the single phase microemulsion region were examined for their capacity to solubilize WHI-P131. A representative ternary phase diagram depicted in FIG. 3, shows the location of the single phase microemulsion region. In this phase diagram, it can be seen that microemulsions containing up to 30% of Captex 300 were possible. These microemulsions were transparent and tolerated dilution very well when mixed with aqueous phases. In WHI-P131-containing microemulsions, the drug was first solubilized in the microemulsions chosen from the one phase region of the phase diagram with mild heating, followed by dilution with water or buffer solution at room temperature.

The microemulsion composition ME1 depicted in Table 9 was used in pharmacokinetic studies and biological activity assays. This microemulsion was prepared by first solubilizing WHI-P131 in composition A in the ternary phase diagram, followed by a dilution with water (1:9). Its volume-weighted average particle diameter as determined by dynamic light scattering was 24.8 nm prior to and 11.4 nm after the incorporation of WHI-P131 chloride. Thus, the drug incorporation, in this case, resulted in the lowering of the particle size. The solubilization of WHI-P131 was at least 1.8 mg per ml of microemulsion. ME2 was a microemulsion composition obtained from a separate phase diagram not shown. This microemulsion can solubilize at least 2.8 mg of WHI-P131 per ml of microemulsion. Compared to the solubility of WHI-P131 salt in the water of 1.2 mg/ml, ME2 had more than doubled the solubilization of WHI-P131 in water. These microemulsions can readily be filtered through 0.2 μm filter, and stored at room temperature. The microemulsions and WHI-P131 they contained were shown to be stable for an extended time at ambient temperature.

By converting WHI-P131 from its free base to its chloride salt form, a fifty fold increase in solubility was achieved raising the drug concentration from 0.025 mg/ml to 1.2 mg/ml. By adding 20% of PEG300 to the vehicle, the drug concentration further increased to 2.2 mg/ml. Furthermore, an incorporation of 3% of PEG2000-DPPE to the cosolvent vehicle brought the drug solubilization to 4.7 mg/ml, which corresponds to a total solubilization enhancement of 190 fold. If a microemulsion formulation instead a cosolvent/micellar solution was used, a total solubilization enhancement of 110 fold. Lead micellar and microemulsion formulations of WHI-P131 were as active as unformulated WHI-P131 in DMSO. The miceller formulation inhibited allergic mast cell responses in vitro and prevented anaphylactic shock in vivo.

These results demonstrates that microemulsions can be used to enhance the solubilization of WHI-P131. However, because of the low solubility of WHI-P131 in the oil, the drug incorporation into the microemulsion seemed to be limited to the surfactant interfacial film only which resulted in a relatively small solubilization enhancement. The lipid cores of the microemulsion droplets, in this case medium chain triglyceride, seemed to contribute very little to the solubilization enhancement.

TABLE 9
Microemulsion compositions containing
WHI-P131

% w/v

| Component | ME1 | ME2 |
|---|---|---|
| WHI-P131 chloride salt | 0.18 | 0.28 |
| Captex 300 | 2.2 | 1.2 |
| Pluronic F-68 | 0.1 | 0.4 |
| Cremophor EL | 1.1 | 1.9 |
| Phospholipon 90G | 1.5 | 1.5 |
| Propylene glycol | 4.7 | 15.3 |
| Purified water | 90.2 | 79.4 |
| Particle size | 10 nm | 15.9 nm |

### Example 11

### Cumulative Solubilization Enhancement

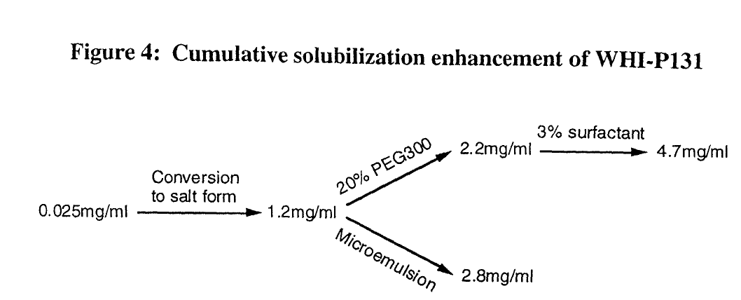

The cumulative solubilization enhancement obtained using a combination of solubilization methods is illustrated in FIG. 4. The overall enhancement appears to be additive. By converting WHI-P131 from its free base to its chloride salt form, a fifty fold increase in solubility was achieved raising the drug concentration from 0.025 mg/ml to 1.2 mg/ml. By adding 20% of PEG300 to the vehicle, the drug concentration further increased to 2.2 mg/ml. Furthermore, an incorporation of 3% of PEG200-DPPE to the cosolvent vehicle brought the drug solubilization to 4.7 mg/ml, which corresponds to a total solubilization enhancement of 190 fold. If a microemulsion formulation instead a cosolvent/micellar solution was used, one can reached a total solubilization enhancement of 110 fold.

### Example 12

### Micellar Formulation for Preclinical Studies in Mice

Preparation of the propylene glycol/surfactant solution

The following materials were weighed into a glass vial: 1.124 g of PEG5000PE, 0.260 g of Pluronic F-68 and 5.704 g of propylene glycol. The mixture was stirred and heated at 70° C. for 5 min or until all the solids were dissolved. The mixture turned into a clear colorless solution. It became solid upon cooling at the room temperature. The mixture was warmed to liquid before use.

WHI-P131 drug containing solution

68 mg of WHI-P131 Cl⁻ was dissolved in 4 ml of the above propylene glycol solution and 0.6 ml DI water. This drug mixture was heated at 70° C. for 10 min until all the WHI-P131 was dissolved and the solution was yellow and clear. This drug solution was mixed into 27.95 ml of DI water dropwise. The diluted solution was yellow and clear. This drug solution was filtered through 0.2 μm filter under a laminar flow hood for sterilization. The filtrate was collected in a liquid scintillation vial. The WHI-P131 concentration in the solution was 1.97 mg/ml. The composition of the solution was:

| Component | Concentration(%) | Concentration Range (%) |
|---|---|---|
| P131 | 0.20 | 0-.21 |
| PEG5000PE | 1.84 | 0.2-2.5 |
| Pluronic F-68 | 0.42 | 0.05-2.0 |
| Propylene glycol | 9.33 | 5.0-20 |
| DI water | 88.21 | Balance |

The control (vehicle) solution

3.74 ml of the propylene glycol solution was mixed with 28.51 ml of DI water. The resulting solution was clear and colorless. This solution was filtered through a 0.2 μm filter under a laminar flow hood for sterilization. The filtrate was collected in a liquid scintillation vial. The composition of the solution was:

| Component | Concentration(%) |
|---|---|
| PEG5000PE | 1.84 |
| Pluronic F-68 | 0.42 |
| Propylene glycol | 9.33 |
| DI water | 88.41 |

### Example 13

### Microemulsion Formulation for Preclinical Studies in Mice

Preparation of low hydrophylicity lipophylicity balance (HBL) phase (100 g):

The following materials were weighed into a 200 ml glass bottle: 2 g of Pluronic F-68, 18 g of Cremophor EL and 80 g of propylene glycol. The mixture was stirred and heated at 70° C. until it turned into a homogeneous suspension.

Preparation of the high HLB phase (100 g):

40 g of Phospholipon 90G and 60 g of Captex 300 were weighted into a 200 ml size glass bottle. The mixture was stirred and heated at 70° C. for several hours until it turned into a clear yellow solution.

Preparation of the Microemulsion (100 g):

In a 200 ml glass bottle, the following components were added: 53.3 g of the high HLB phase, 33.3% of the HLB phase and 13.3 g of DI water. The bottle was hand shaken until the mixture became a transparent microemulsion.

Preparation of P131 Drug Microemulsion (96 ml of 0.20% WHI-P131 Solution:

220 mg of WHI-P131 was dissolved in 15.7 ml of the above microemulsion. The mixture was stirred and heated at 70° C. for 30 min or until all solids were dissolved. WHI-P131 concentration in this drug microemulsion was 14 mg/ml.

14 ml of this drug microemulsion was mixed into 84 ml DI water dropwise. WHI-P131 concentration in this solution was 2.0 mg/ml. The composition of the solution was shown in the following table:

| Component | Concentration(%) | Concentration Range (%) |
|---|---|---|
| WHI-P131 | 0.20 | 0-.22 |
| Pluronic F-68 | 0.15 | 0.05-2.0 |
| Cremophor EL | 1.37 | 1.2-1.3 |
| Propylene glycol | 6.09 | 2.0-12 |
| Phospholipon 90G | 1.90 | 0.50-2.8 |
| Captex 300 | 1.90 | 0.75-4.2 |
| DI water | Balance | Balance |

### Example 14

### Pharmacokinetic Study

Pharmacokinetic studies:

In pharmacokinetic studies, mice were injected intravenously via the tail vein with a bolus dose of 300 μg/mouse (˜12.5 mg/kg=34 μmoles/kg) of WHI-P131. Blood samples were obtained from the ocular venous plexus by retroorbital venupuncture prior to and at 3, 5, 10, 15, 30, 45 minutes, and 1, 2, 4, and 8 hours after administration of WHI-P131. All collected blood samples were heparinized and centrifuged at 7,000 g for 10 min in a microcentrifuge to obtain plasma. The plasma samples were stored at −20° C. until analysis. Aliquots of plasma were used for extraction and HPLC analysis. Pharmacokinetic modeling and parameter calculations were carried out using the software, WinNonlin Program, Version 2.0. An appropriate pharmacokinetic model was chosen on the basis of lowest weighted squared residuals, lowest Schwartz criterion, lowest Akaike's Information Criterion value, lowest standard errors of the fitted parameters, and dispersion of the residuals. The elimination half-life was estimated by linear regression analysis of the terminal phase of the plasma concentration profile. The area under the curve (AUC) was calculated by the trapezoidal rule between first (0 h) and last sampling time plus C/k, where C is the concentration of last sampling and k is the elimination rate constant. Systemic clearance (CLs) was determined by dividing the dose by the AUC. Statistical analysis was performed using the Instat program, 3.0. The significance of differences between pharmacokinetic parameters was analyzed using two-tailed t test, and P values <0.05 were considered significant.

A highly sensitive quantitative HPLC detection method was used to determine the pharmacokinetics of WHI-P131. In brief, the HPLC system consisted of a Hewlett Packard series 1100 equipped with an automated electronic degasser, a quaternary pump, an autosampler, an automatic thermostatic column compartment, diode array detector and a computer with a Chemstation software program for data analysis. A 250×4 mm Lichrospher 100, RP-18 (5 μm) analytical and a 4×4 mm Lichrospher 100, RP-18 guard columns were obtained from Hewlett Packard Inc. Acetonitrile/water containing 0.1% of trifluoroacetic acid and 0.1% triethylamine (28:72, v/v) was used as the mobile phase. The wavelength of detection was set at 340 nm. Peak width, response time and slit were set at >0.03 min, 0.5 s and 8 nm, respectively.

For determination of WHI-P131 levels, 10 μL of internal standard was added to a 100 μL plasma sample. For extraction, 7 ml chloroform was then added to the plasma sample, and the mixture was vortexed thoroughly for 3 min. Following centrifugation (300 g, 5 min), the aqueous layer was frozen using acetone/dry ice and the organic phase was transferred into a clean test tube. The chloroform extracts were dried under a slow steady stream of nitrogen. The residue was reconstituted in 100 μL of methanol: water (9:1) and 50 μL aliquot of this solution was used for HPLC analysis. Under the described chromatographic separation conditions, the retention times for WHI-P131 and the internal standard were 5.1 minutes and 9.5 minutes, respectively. At the retention time, WHI-P131 and its internal standard were eluted without any interference peaks from the blank plasma. The plasma calibration standards were linear in 0.1-20 μM range. The coefficient of variation for within the day and from day-to-day was <10%. The linear coefficient of determination was greater than 0.999. The lower limit of detection was 0.05 μM and the mean accuracy of quality control samples was between 90-110% for all analysis days.

Mast Cell Inhibition Assay:

RBL-2H3 mast cell line was obtained from Dr. Reuben P. Siraganian (Laboratory of Microbiology and Immunology, National Institute of Dental Research, National Institute of Health). The cells were maintained as monolayer cultures in 75- or 150-cm² flask in Eagle's essential medium supplemented with 20% fetal calf serum (Hamawy et. al., 1995, Cellular Signalling 7:535-544). RBL-2H3 cells were sensitized with monoclonal anti-DNP IgE antibody (0.24 mg/ml) for 1 hour at 37° C. in a 48-well tissue culture plate. RBL-2H3 cells were allowed to adhere to the plate. Unbound IgE was removed by washing the cells with phosphate buffered saline. After washing, PIPES-buffered saline containing 1 mM calcium chloride was added to the monolayers of the RBL-2H3 cells. The cells were challenged with 20 ng/ml DNP-BSA for 30 minutes at 37° C. The plate was centrifuged at 200 g for 10 minutes at 4° C. Supernatants were removed and saved. β-hexosaminidase release was estimated in cell free supernatants and 0.1% Triton X-100 solubilized pellets, as described (Malaviya R et al., J Biol Chem., 1999, 274, 27028-38; Ozawa et. al., 1993, J Biol. Chem., 268:1749-1756).

Anaphylaxis Model:

In the murine model for antigen induced active anaphylaxis (Malaviya R et al., Targeting Janus kinase 3 in mast cells prevents immediate hypersensitivity reactions and anaphylaxis. J Biol Chem., 1999, 274, 27028-38), mice were sensitized with 2 mg BSA in 200 μl aluminum hydroxide gel (Reheis Inc., Berkeley, N.J.), which induces the production of IgE response to the presented antigen. Ten days later anaphylactic shock was induced by the i.v. injection of the animals with 200 μg BSA. Mice were continuously monitored for 3 hours for signs of anaphylaxis.

Mice

Male Balb/c mice (6-8 weeks old) were purchased from Charles River Laboratories (Wilmington, Mass.). Breeder pairs of JAK3-null mice (Nosaka et. al., 1995) were obtained from Dr. J. Ihle (St. Jude Children's Research Hospital, Memphis, Tenn.). Animals were caged in groups of five in a pathogen free environment in accordance with the rules and regulations of U.S. Animal Welfare Act, and National Institutes of Health (NIH). Animal care and the experimental procedures were carried out in agreement with institutional guidelines.

Study

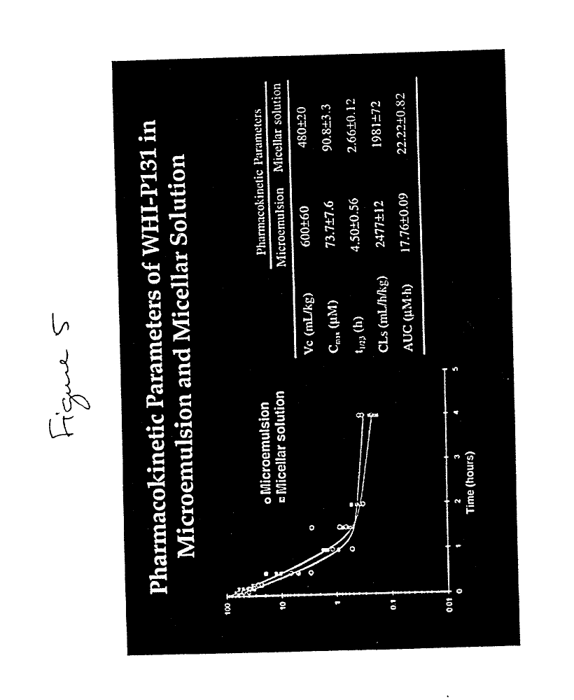

We compared the pharmacokinetics of the lead micellar microemulsion formulations of WHI-P131. The WHI-P131 plasma concentration-time curves following i.v. bolus injection of WHI-P131 formulations in mice are depicted in FIG. 5. It shows that the plasma concentration time curves for the two vehicles were practically superimposable. When pharmacokinetic calculations were made, a two compartment first order pharmacokinetic model was found to give the best fit for the plasma concentration versus time curves. A summary of pharmacokinetic parameters of of the two WHI-P131 formulations, obtained using the afore-mentioned models and software programs, shows that the maximum plasma concentrations Cmax attained at the fixed WHI-P131 dose level of 13 mg/kg were very similar. In addition, the systemic exposure levels, as measured by the AUC, were also similar.

Dynamic light scattering spectroscopy has shown that the WHI-P131 containing microemulsions had a mean particle size of 10-25 nm, whereas micellar solutions had particle size well below 10 nm. Both micellar and microemulsion formulations of WHI-P131 are biologically active and have similar pharmacokinetic profiles in vivo.

### Example 15

### Mast Cell Inhibitory “Anti-allergic” Activity of Formulated WHI-P131 in vitro

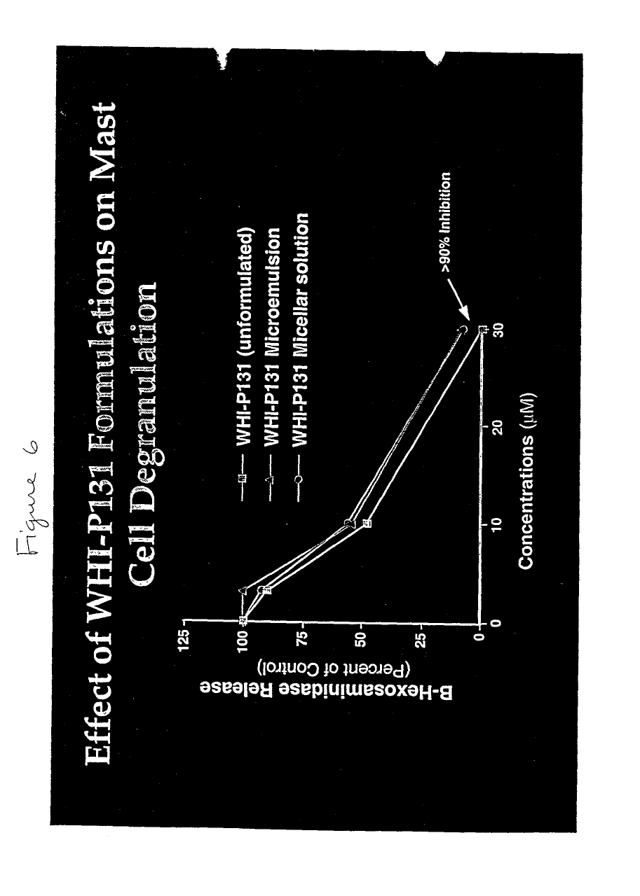

Micellar solution and microemulsion formulations of WHI-P131 were active. FIG. 6 shows the mast cell inhibitory “anti-allergic” activity of these formulations in vitro. Mast cell degranulation (β-hexosaminidase release, % of total), was assessed by measuring the β-hexosaminidase levels in cell free supernatants and Triton X-100 solubilized pellets using the formula: β-hexosaminidase release, % of total=100× (β-hexosaminidase level in supernatant/β-hexosaminidase level in supernatant+solubilized pellet). Unformulated WHI-P131 has been previously shown to prevent mast cell degranulation and release of preformed granule-associated β-hexosaminidase in a dose-dependent fashion with near to complete inhibition at ≧30 μM (Malaviya R et al., Targeting Janus kinase 3 in mast cells prevents immediate hypersensitivity reactions and anaphylaxis. J Biol Chem., 1999, 274, 27028-38). As shown in FIG. 6, both formulations were as effective as unformulated WHIP131 in DMSO. Virtually complete inhibition of mast cell function was achieved at a WHI-P131 concentration of 30 μM.

### Example 16

### In vivo anti-allergic activity formulated WHI-P131

We tested the efficacy of WHI-P131 in a model of IgE/antigen-induced active systemic anaphylaxis. To this end, mice were first injected with BSA in an aluminum hydroxide gel to trigger a BSA-specific IgE response. Ten days later, these BSA-sensitized mice were rechallenged with this antigen to induce anaphylaxis. Only one of 20 (5%) saline treated control mice and 4 of 25 (16%) micelle vehicle (0% WHI-P131) treated control mice did not develop fatal anaphylaxis (Table 10). The remainder of these control mice (i.e., 40 of 45) developed anaphylaxis and died within 45 min after antigen challenge. In contrast, 7 of 10 (70%) BSA-sensitized mice that were treated with WHI-P131 (micellar formulation) prior to antigen challenge survived without any signs of anaphylaxis, (P<0.05 by log-rank test).

TABLE 10
Protective activity of the WHI-P131 Micellar
Formulation against Active Anaphylaxis in Mice.

| | | Number of | |
|---|---|---|---|
| | Number of Mice | Mice | |
| Treatment Groups | Tested | Survived | Percent Survival |
| Saline Control | 20 | 1 | 5 |
| Micelle vehicle | 25 | 4 | 16 |
| WHI-P131-Micelle | 10 | 7 | 70 |

To study the effect of WHI-P131 formulations on fatal anaphylaxis in mice, BALB/c mice were sensitized with 100 mg/kg bovine serum albumin in 200 μl of the adjuvant aluminum hydroxide gel (Reheis Inc., Berkeley, N.J.), which favors the production of IgE in response to the presented antigen. Ten days later, mice were treated with two doses of WHI-P131 formulations (50 mg/kg) or vehicle intraperitoneally 10 min before and 10 min after an intravenous injection of the 10 mg/kg BSA. Mice were continuously monitored for 3 hours for signs of anaphylaxis and the mice surviving the anaphylactic reaction were sacrificed.

FIG. 6 shows effects of WHI-P131 formulations on IgE receptor/Fc epsilon RI-mediated mast cell degranulation. RBL-2H3 cells were sensitized with monoclonal anti-DNP IgE, treated with WHI-P131 formulations or vehicle control compounds for 1 h, and then challenged with 20 ng/ml DNP-BSA for 30 min. Mast cell degranulation (β-hexosaminidase release, % of total) was assessed by measuring the β-hexosaminidase levels in cell free supernatants and Triton X-100 solubilized pellets using the formula: β-hexosaminidase release, % of total=100×(β-hexosaminidase level in supernatant/β-hexosaminidase level in supernatant+solubilized pellet). Vehicle treated control RBL-2H3 cells released 37.1±4.3% of their hexosaminidase contents after DNP-BSA challenge. The data points represent the mean±SEM values obtained from 3-4 independent experiments.

All publications, patents, and patent documents described herein are incorporated by reference as if fully set forth. The invention described herein may be modified to include alternative embodiments. All such obvious alternatives are within the spirit and scope of the invention, as claimed below.

We claim:

1. A formulation comprising:
(a) a phospholipid; and
(b) a quinazoline compound of the formula: 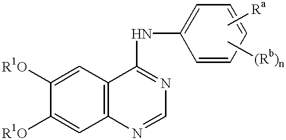
wherein:
R^{a} is hydrogen, halo, hydroxy, mercapto, (C₁-C₄)hydroxyalkyl, methylenedioxy, ethylenedioxy, benzyloxy, OCF₃, SCF₃, SO₃H, SO₂F, SO₂NR²R³ in which R² is hydrogen or (C₁-C₄)alkyl and R³ is hydrogen, (C₁-C₄)alkyl, or phenyl, NR²R⁴ in which R² is as defined above and R⁴ is phenyl, or R^{a} a group of the formula: 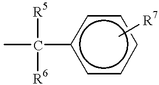
in which R⁵ and R⁶ are each, independently, hydrogen, (C₁-C₄)alkyl, or (C₁-C₄)perfluoroalkyl, and R⁷ is hydrogen, halo, hydroxy, (C₁-C₄)alkyl, (C₁-C₄)alkoxy, (C₁-C₄)hydroxyalkyl, or N(R²)₂ in which R² is as defined above;
n is an integer of 1-4;
R^{b} is each, independently, hydrogen, halo, hydroxy, mercapto, (C₁-C₄)alkyl, (C₁-C₄)alkoxy, (C₁-C₄)thioalkyl, (C₁-C₄)hydroxyalkyl, nitro, cyano, methylenedioxy, ethylenedioxy, COCH₃, CF₃, OCF₃, SCF₃, COOH, SO₃H, SO₂F, phenyl or phenyl substituted by a group selected from halo, hydroxy, mercapto, (C₁-C₄)alkyl, (C₁-C₄)alkoxy, (C₁-C₄)thioalkyl, (C₁-C₄)hydroxyalky], amino, nitro, cyano, CF₃, COOH, SO₃H, SO₂NR²R³ in which R² and R³ are as defined below, and SO₂F;
R^{a} is also benzyloxy substituted on the phenyl portion by a group defined above, NR²R³ in which R² is H or (C₁-C₄)alkyl and R³ is H, (C₁-C₄)alkyl, phenyl or phenyl substituted by a group as defined above;
R¹ is (C₁-C₄)alkyl, or a pharmaceutically acceptable salt thereof.

2. The formulation of claim 1, wherein the quinazoline compound is an acid addition salt.

3. The formulation of claim 1, wherein R¹ is methyl.

4. The formulation of claim 1, wherein the quinazoline compound is selected from:
4-(3′,5′-dibromo-4′-methylphenyl)amino-6,7-dimethoxyquinazoline,
4-(2′,4′,6′-tribromophenyl)amino-6,7-dimethoxyquinazoline,
4-(2′,3′,5′,6′-tetrafluoro-4′-bromophenyl)amino-6,7-dimethoxyquinazoline,
4-(4′-fluorophenyl)amino-6,7-dimethoxyquinazoline,
4-(3′-fluorophenyl)amino-6,7-dimethoxyquinazoline,
4-(2′-fluorophenyl)amino-6,7-dimethoxyquinazoline,
4-(4′-trifluoromethylphenyl)amino-6,7-dimethoxyquinazoline,
4-(2′-trifluoromethylphenyl)amino-6,7- dimethoxyquinazoline,
4-(3′,5′-bis-trifluoromethylphenyl)amino-6,7-dimethoxyquinazoline,
4-(3′,5′-dibromo-4′-hydroxyphenyl)amino-6,7-dimethoxyquinazoline, and
4-(3′-chloro-4′-hydroxyphenyl)amino-6,7-dimethoxyquinazoline.

5. The formulation of claim 1, wherein the quinazoline compound is selected from:
4-(3′,5′-dibromo-4′-hydroxyphenyl)amino-6,7-dimethoxyquinazoline,
4-(4′-hydroxyphenyl)amino-6,7-dimethoxyquinazoline,
4-(3′-hydroxyphenyl)amino-6,7-dimethoxyquinazoline,
4-(2′-hydroxy-naphthyl-3′)-amino-6,7-dimethoxyquinazoline,
4-{4′-[2″-(4″′-aminophenyl)-hexafluoropropyl]phenyl}-amino-6,7-dimethoxyquinazoline, and
4-(3′-trifluoromethoxylphenyl)-amino-6,7-dimethoxyquinazoline.

6. The formulation of claim 1, wherein the phospholipid is an unsaturated phospholipid.

7. The formulation of claim 1, wherein the phospholipid is an anionic phospholipid.

8. The formulation of claim 1, wherein the phospholipid is a polyethylene glycol phosholipid.

9. The formulation of claim 1, wherein the phospholipid is a polyethylene glycol phosphatidylethanolamine.

10. The formulation of claim 1, wherein the phosholipid is 1,2-dipalmitoyl-sn-glycero-3-phosphoethanolamine-N-[poly(ethylene glycol)5000].

11. The formulation of claim 1, wherein the phosholipid is 1,2-dipalmitoyl-sn-glycero-3-phosphoethanolamine-N-[poly(ethylene glycol)2000].

12. The formulation of claim 1, further comprising a surfactant.

13. The formulation of claim 11, wherein the surfactant is a b lock copolymer of ethyleneoxide and propyleneoxide.

14. The formulation of claim 1, further comprising propylene glycol.

15. The formulation of claim 1, further comprising:
(c) a surfactant
(d) propylene glycol and
(e) water.

16. The formulation of claim 15, wherein the phospholipid is polyethylene glycol phosphatidylethanolamine and the surfactant is a block copolymer of ethyleneoxide and propyleneoxide.

17. The formulation of claim 15, wherein the phospholipid is an anionic phospholipid and the quinazoline compound is a cationic quinazoline compound.

18. The formulation of claim 15, wherein:
(a) the phospholipid concentration is about 0.2 to 2.5 w/v %;
(b) the quinazoline concentration is less than about 0.2 w/v %;
(c) the surfactant concentration is about 0.05-2 w/v %;
(d) the propylene glycol concentration is about 5-20 w/v %; and
(e) the balance is water.

19. The formulation of claim 18, wherein:
(a) the phospholipid concentration is about 1.84 w/v %;
(b) the quinazoline concentration is about 0.2 w/v %;
(c) the surfactant concentration is about 0.42 w/v %;
(d) the propylene glycol concentration is about 9.33 w/v %; and
(e) the water concentration is 88.21.

20. The formulation of claim 1, wherein the phospholipid and quinazoline compound form a micellar formulation with a mean particle size less than about 10 nm.

21. The formulation of claim 15, wherein the phospholipid and quinazoline compound form a micellar formulation with a mean particle size less than about 10 nm.

22. A formulation comprising:
(a) a low hydrophylicity lipophylicity balance portion comprising:
(i) a block copolymer of ethylene oxide and propylene oxide;
(ii) an ethoxylated castor oil;
(iii) propylene glycol;
(b) a high hydrophylicity lipophylicity balance portion comprising:
(i) lecithin;
(ii) a triglyceride of caprylic acid;
(c) water; and
(d) a quinazoline compound of the formula: 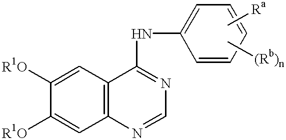
wherein:
R^{a} is hydrogen, halo, hydroxy, mercapto, (C₁-C₄)hydroxyalkyl, methylenedioxy, ethylenedioxy, benzyloxy, OCF₃, SCF₃, SO₃H, SO₂F, SO₂NR²R³ in which R² is hydrogen or (C₁-C₄)alkyl and R³ is hydrogen, (C₁-C₄)alkyl, or phenyl, NR²R⁴ in which R² is as defined above and R⁴ is phenyl, or R^{a} a group of the formula: 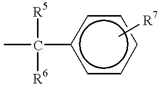
in which R⁵ and R⁶ are each, independently, hydrogen, (C₁-C₄)alkyl, or (C₁-C₄)perfluoroalkyl, and R⁷ is hydrogen, halo, hydroxy, (C₁-C₄)alkyl, (C₁-C₄)alkoxy, (C₁-C₄)hydroxyalkyl, or N(R²)₂ in which R² is as defined above;
n is an integer of 1-4;
R^{b} is each, independently, hydrogen, halo, hydroxy, mercapto, (C₁-C₄)alkyl, (C₁-C₄)alkoxy, (C₁-C₄)thioalkyl, (C₁-C₄)hydroxyalkyl, nitro, cyano, methylenedioxy, ethylenedioxy, COCH₃, CF₃, OCF₃, SCF₃, COOH, SO₃H, SO₂F, phenyl or phenyl substituted by a group selected from halo, hydroxy, mercapto, (C₁-C₄)alkyl, (C₁-C₄)alkoxy, (C₁-C₄)thioalkyl, (C₁-C₄)hydroxyalkyl, amino, nitro, cyano, CF₃, COOH, SO₃H, SO₂NR²R³ in which R² and R³ are as defined below, and SO₂F;
R^{a} is also benzyloxy substituted on the phenyl portion by a group defined above, NR²R³ in which R² is H or (C₁-C₄)alkyl and R³ is H, (C₁-C₄)alkyl, phenyl or phenyl substituted by a group as defined above;
R¹ is (C₁-C₄)alkyl, or a pharmaceutically acceptable salt thereof.

23. The formulation of claim 22, wherein the low hydrophylicity lipophylicity balance portion, the high hydrophylicity lipophylicity balance portion, the water and the quinazoline compound form a microemulsion with a mean particle size of about 10-25 nm.

24. The formulation of claim 22, wherein the low hydrophylicity lipophylicity balance portion comprises:
(i) about 2 w/v % of the block copolymer of ethylene oxide and propylene oxide;
(ii) about 18 w/v % of the ethoxylated castor oil; and
(iii) about 80 w/v % of the propylene glycol.

25. The formulation of claim 22, wherein the high hydrophylicity lipophylicity balance portion comprises:
(i) about 40 w/v % of the lecithin; and
(ii) about 60 w/v % of the triglyceride of caprylic acid.

26. The formulation of claim 22, wherein the low hydrophylicity lipophylicity balance portion comprises:
(i) about 2 w/v % of the block copolymer of ethylene oxide and propylene oxide;
(ii) about 18 w/v % of the ethoxylated castor oil;
(iii) about 80 w/v % of the propylene glycol; and
the high hydrophylicity lipophylicity balance portion comprises:
(i) about 40 w/v % of the lecithin;
(ii) about 60 w/v % of the triglyceride of caprylic acid;
the water and the quinazoline compound form a microemulsion with a mean particle size of about 10-25 nm.

27. The formulation of claim 22, wherein the quinazoline compound is an acid addition salt.

28. The formulation of claim 22, wherein the quinazoline compound is selected from:
4-(3′,5′-dibromo-4′-methylphenyl)amino-6,7-dimethoxyquinazoline,
4-(2′,4′,6′-tribromophenyl)amino-6,7-dimethoxyquinazoline,
4-(2′,3′,5′,6′-tetrafluoro-4′-bromophenyl)amino-6,7-dimethoxyquinazoline,
4-(4′-fluorophenyl)amino-6,7-dimethoxyquinazoline,
4-(3′-fluorophenyl)amino-6,7-dimethoxyquinazoline,
4-(2′-fluorophenyl)amino-6,7-dimethoxyquinazoline,
4-(4′-trifluoromethylphenyl)amino-6,7-dimethoxyquinazoline,
4-(2′-trifluoromethylphenyl)amino-6,7-dimethoxyquinazoline,
4-(3′,5′-bis-trifluoromethylphenyl)amino-6,7-dimethoxyquinazoline,
4-(3′,5′-dibromo-4′-hydroxyphenyl)amino-6,7-dimethoxyquinazoline, and
4-(3′-chloro-4′-hydroxyphenyl)amino-6,7-dimethoxyquinazoline.

29. The formulation of claim 22, wherein the quinazoline compound is selected from:
4-(3′,5′-dibromo-4′-hydroxyphenyl)amino-6,7-dimethoxyquinazoline,
4-(4′-hydroxyphenyl)amino-6,7-dimethoxyquinazoline,
4-(3′-hydroxyphenyl)amino-6,7-dimethoxyquinazoline,
4-(2′-hydroxy-naphthyl-3′)-amino-6,7-dimethoxyquinazoline,
4-{4′-[2″-(4″′-aminophenyl)-hexafluoropropyl]phenyl}-amino-6,7-dimethoxyquinazoline, and
4-(3′-trifluoromethoxylphenyl)-amino-6,7-dimethoxyquinazoline.

30. A method of making a formulation comprising:
(a) providing a quinazoline compound of the formula: 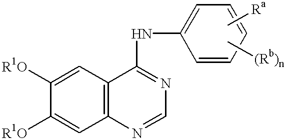
wherein:
R^{a} is hydrogen, halo, hydroxy, mercapto, (C₁-C₄)hydroxyalkyl, methylenedioxy, ethylenedioxy, benzyloxy, OCF₃, SCF₃, SO₃H, SO₂F, SO₂NR²R³ in which R² is hydrogen or (C₁-C₄)alkyl and R³ is hydrogen, (C₁-C₄)alkyl, or phenyl, NR²R⁴ in which R² is as defined above and R⁴ is phenyl, or R^{a} a group of the formula: 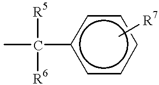
in which R⁵ and R⁶ are each, independently, hydrogen, (C₁-C₄)alkyl, or (C₁-C₄)perfluoroalkyl, and R⁷ is hydrogen, halo, hydroxy, (C₁-C₄)alkyl, (C₁-C₄)alkoxy, (C₁-C₄)hydroxyalkyl, or N(R²)₂ in which R² is as defined above;
n is an integer of 1-4;
R^{b} is each, independently, hydrogen, halo, hydroxy, mercapto, (C₁-C₄)alkyl, (C₁-C₄)alkoxy, (C₁-C₄)thioalkyl, (C₁-C₄)hydroxyalkyl, nitro, cyano, methylenedioxy, ethylenedioxy, COCH₃, CF₃, OCF₃, SCF₃, COOH, SO₃H, SO₂F, phenyl or phenyl substituted by a group selected from halo, hydroxy, mercapto, (C₁-C₄)alkyl, (C₁-C₄)alkoxy, (C₁-C₄)thioalkyl, (C₁-C₄)hydroxyalkyl, amino, nitro, cyano, CF₃, COOH, SO₃H, SO₂NR²R³ in which R² and R³ are as defined below, and SO₂F;
R^{a} is also benzyloxy substituted on the phenyl portion by a group defined above, NR²R³ in which R² is H or (C₁-C₄)alkyl and R³ is H, (C₁-C₄)alkyl, phenyl or phenyl substituted by a group as defined above;
R¹ is (C₁-C₄)alkyl, or a pharmaceutically acceptable salt thereof;
the quinazoline compounding having a first solubility in water;
(b) converting the quinazoline compound to an acid addition salt of the quinazoline compound having a second solubility in water greater than the first solubility in water;
(c) combining polyethylene glycol with the acid addition salt of the quinazoline compound to form a first mixture, the first mixture having a third solubility of quinazoline compound in water/polyethylene glycol greater than the second solubility in water;
(d) combining a phospholipid with the first mixture to form a second mixture, the second mixture having a fourth solubility of quinazoline compound in water/polyethylene glycol/phospholipid greater than the third solubility in water/polyethylene glycol.

31. The method of claim 30, wherein the second solubility is at least about 50 times greater than the first solubility.

32. The method of claim 30, wherein the third solubility is at least about 90 times greater than the first solubility.

33. The method of claim 30, wherein the fourth solubility is at least about 190 times greater than the first solubility.

34. The method of claim 30, wherein the quinazoline compound is selected from:
4-(3′,5′-dibromo-4′-methylphenyl)amino-6,7-dimethoxyquinazoline,
4-(2′,4′,6′-tribromophenyl)amino-6,7-dimethoxyquinazoline,
4(2′,3′,5′,6′-tetrafluoro-4′-bromophenyl)amino-6,7-dimethoxyquinazoline,
4-(4′-fluorophenyl)amino-6,7-dimethoxyquinazoline,
4-(3′-fluorophenyl)amino-6,7-dimethoxyquinazoline,
4-(2′-fluorophenyl)amino-6,7-dimethoxyquinazoline,
4-(4′-trifluoromethylphenyl)amino-6,7-dimethoxyquinazoline,
4-(2′-trifluoromethylphenyl)amino-6,7-dimethoxyquinazoline,
4-(3′,5′-bis-trifluoromethylphenyl)amino-6,7-dimethoxyquinazoline,
4-(3′,5′-dibromo-4′-hydroxyphenyl)amino-6,7-dimethoxyquinazoline, and
4-(3′-chloro-4′-hydroxyphenyl)amino-6,7-dimethoxyquinazoline.

35. The method of claim 30, wherein the quinazoline compound is selected from:
4-(3′,5′-dibromo-4′-hydroxyphenyl)amino-6,7-dimethoxyquinazoline,
4-(4′-hydroxyphenyl)amino-6,7-dimethoxyquinazoline,
4-(3′-hydroxyphenyl)amino-6,7-dimethoxyquinazoline,
4-(2′-hydroxy-naphthyl-3′)-amino-6,7-dimethoxyquinazoline,
4-{4′-[2″-(4″′-aminophenyl)-hexafluoropropyl]phenyl}-amino-6,7-dimethoxyquinazoline, and
4-(3′-trifluoromethoxylphenyl)-amino-6,7-dimethoxyquinazoline.

36. The method of claim 30, wherein the phospholipid is an unsaturated phospholipid.

37. The method of claim 30, wherein the phospholipid is an anionic phospholipid.

38. The method of claim 30, wherein the phospholipid is a polyethylene glycol phosholipid.

39. The method of claim 30, wherein the phospholipid is a polyethylene glycol phosphatidylethanolamine.

40. The method of claim 30, wherein the phosholipid is 1,2-dipalmitoyl-sn-glycero-3-phosphoethanolamine-N-[poly(ethylene glycol)5000].

41. The method of claim 30, wherein the phosholipid is 1,2-dipalmitoyl-sn-glycero-3-phosphoethanolamine-N-[poly(ethylene glycol)2000].

42. A product produced by the method of claim 30.

43. A method comprising, administering to a mammal a formulation comprising:
(a) a phospholipid; and
(b) a mast cell inhibiting amount of 4-(4′-hydroxyphenyl)amino-6,7-dimethoxyquinazoline.

44. The method of claim 43, wherein the phospholipid is an unsaturated phospholipid.

45. The method of claim 43, wherein the phospholipid is an anionic phospholipid.

46. The method of claim 43, wherein the phospholipid is a polyethylene glycol phosholipid.

47. The method of claim 43, wherein the phospholipid is a polyethylene glycol phosphatidylethanolamine.

48. The method of claim 43, wherein the phosholipid is 1,2-dipalmitoyl-sn-glycero-3-phosphoethanolamine-N-[poly(ethylene glycol)5000].

49. The method of claim 43, wherein the phosholipid is 1,2-dipalmitoyl-sn-glycero-3-phosphoethanolamine-N-[poly(ethylene glycol)2000].

50. The method of claim 43, further comprising a surfactant.

51. The method of claim 43, wherein the surfactant is a block copolymer of ethyleneoxide and propyleneoxide.

52. The method of claim 43, further comprising propylene glycol.

53. The method of claim 43, further comprising:
(c) a surfactant
(d) propylene glycol and
(e) water.

54. The method of claim 43, wherein the phospholipid is polyethylene glycol phosphatidylethanolamine and the surfactant is a block copolymer of ethyleneoxide and propyleneoxide.

55. The method of claim 43, wherein the phospholipid is an anionic phospholipid and the 4-(4′-hydroxyphenyl)amino-6,7-dimethoxyquinazoline compound is a chloride salt of 4-(4′-hydroxyphenyl)amino-6,7-dimethoxyquinazoline.

56. The method of claim 43, wherein:
(a) the phospholipid concentration is about 0.2 to 2.5 w/v %;
(b) the 4-(4′-hydroxyphenyl)amino-6,7-dimethoxyquinazoline concentration is less than about 0.2 w/v %;
(c) the surfactant concentration is about 0.05-2 w/v %;
(d) the propylene glycol concentration is about 5-20 w/v %; and
(e) the balance is water.

57. The method of claim 43, wherein:
(a) the phospholipid concentration is about 1.84 w/v %;
(b) the 4-(4′-hydroxyphenyl)amino-6,7-dimethoxyquinazoline concentration is about 0.2 w/v %;
(c) the surfactant concentration is about 0.42 w/v %;
(d) the propylene glycol concentration is about 9.33 w/v %; and
(e) the water concentration is 88.21.

58. The method of claim 43, wherein the phospholipid and 4-(4′-hydroxyphenyl)amino-6,7-dimethoxyquinazoline form a micellar formulation with a mean particle size less than about 10 nm.

59. The method of claim 53, wherein the phospholipid and 4-(4′-hydroxyphenyl)amino-6,7-dimethoxyquinazoline form a micellar formulation with a mean particle size less than about 10 nm.

60. A method comprising, administering to a mammal a formulation comprising:
(a) a low hydrophylicity lipophylicity balance portion comprising:
(i) a block copolymer of ethylene oxide and propylene oxide;
(ii) an ethoxylated castor oil;
(iii) propylene glycol;
(b) a high hydrophylicity lipophylicity balance portion comprising:
(i) lecithin;
(ii) a triglyceride of caprylic acid;
(c) water; and
(d) a mast cell inhibiting amount of 4-(4′-hydroxyphenyl)amino-6,7-dimethoxyquinazoline.

61. The method of claim 60, wherein the low hydrophylicity lipophylicity balance portion, the high hydrophylicity lipophylicity balance portion, the water and the mast cell inhibiting amount of 4-(4′-hydroxyphenyl)amino-6,7-dimethoxyquinazoline form a microemulsion with a mean particle size of about 10-25 nm.

62. The method of claim 60, wherein the low hydrophylicity lipophylicity balance portion comprises:
(i) about 2 w/v % of the block copolymer of ethylene oxide and propylene oxide;
(ii) about 18 w/v % of the ethoxylated castor oil; and
(iii) about 80 w/v % of the propylene glycol.

63. The method of claim 60, wherein the high hydrophylicity lipophylicity balance portion comprises:
(i) about 40 w/v % of the lecithin; and
(ii) about 60 w/v % of the triglyceride of caprylic acid.

64. The method of claim 60, wherein the low hydrophylicity lipophylicity balance portion comprises:
(i) about 2 w/v % of the block copolymer of ethylene oxide and propylene oxide;
(ii) about 18 w/v % of the ethoxylated castor oil;
(iii) about 80 w/v % of the propylene glycol; and
the high hydrophylicity lipophylicity balance portion comprises:
(i) about 40 w/v % of the lecithin;
(ii) about 60 w/v % of the triglyceride of caprylic acid;
the water and the quinazoline compound form a microemulsion with a mean particle size of about 10-25 nm.

65. The method of claim 60, wherein the mast cell inhibiting amount of 4-(4′-hydroxyphenyl)amino-6,7-dimethoxyquinazoline is an acid addition salt of 4-(4′-hydroxyphenyl)amino-6,7-dimethoxyquinazoline.
